(19) United States
(12) Patent Application Publication
(10) Pub. No.: US 2005/0042316 A1
(43) Pub. Date: Feb. 24, 2005

(54) Attractants for moths

(76) Inventors: Peter Charles Gregg, New South Wales (AU); Alice Perez Del Socorro, New South Wales (AU)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/476,621

(22) PCT Filed: May 3, 2002
(86) PCT No.: PCT/AU02/00554

(30) Foreign Application Priority Data
May 3, 2001 (AU) PR 4797

(51) Int. Cl. A61K 35/78; A61K 31/045; A61K 31/11

(52) U.S. Cl. 424/778; 514/699; 514/729; 514/730

(57) ABSTRACT

A method of selecting the components of a blend attractive to moth pests, comprising the steps of: (1) measuring the attractiveness of a plurality of single candidate compounds; and (2) selecting for blending those candidate compounds which show statistically significant attraction.

### TECHNICAL FIELD

The present invention is concerned with attractants for adult moths, particularly those in the family Noctuidae and, more particularly, those of Helicoverpa species.

### BACKGROUND ART

It has long been appreciated that it would be desirable to attract insect pests to a locus, where action may be taken either to kill the pest or to otherwise reduce its numbers. This strategy is referred to as an “attract-and-kill” strategy. Pheromone attractants have been previously used in attract-and-kill strategies, however complications associated with variation in sex ratios, multiple mating, female competition, immigration of mated females and male responsiveness to pheromones make the effectiveness of this strategy uncertain (Gregg & Wilson 1991). Nevertheless, in cotton, the attract-and-kill approach using pheromones has led to significant reductions in boll weevil populations in the United States of America (Smith et al. 1994) and in pink bollworm populations in Egypt (Mafra-Neto and Habib 1996). Attract-and-kill methods using crude bait such as molasses were commonly used for Helicoverpa zea in the United States of America before the development of synthetic insecticides (for example, Ditman 1937). It is nevertheless a considerable disadvantage that pheromone attractants attract only male moths, and crude preparations have limited effectiveness.

Dissemination of selective pathogens of pest moth species is potentially another means for control. In such a technique, moths would be lured to a trap, contaminated with the pathogen, and then released. This might be particularly valuable with the new generation of genetically modified organisms which kill the hosts quickly, without the normal increase in inoculum which accompanies an epidemic. However, success of such a technique relies upon having available an effective attractant for such pests.

A further means of reducing pest moth numbers which has been proposed is the use of trap cropping. Trap cropping is becoming widely used in the cotton industry, with the most common trap crops being chick peas in spring and pigeon peas in summer and autumn. Helicoverpa females are attracted to an area of trap crop where they remain and oviposit. The trap crop is then destroyed and the insect eggs are destroyed along with it. While this technique to date has relied upon the natural attractiveness of the crop, if an effective attractant for female pest moths were available the efficacy of this technique may be greatly increased.

It will therefore be appreciated that there is a substantial need for an effective attractant of pest moth species, which attracts females as well as males.

It has long been observed that certain insects feed preferentially upon selected plants. Therefore, there have been a number of attempts to develop attractants based upon the volatile components of such plants which seek to duplicate the olfactory profile of an attractive plant. For example, in U.S. Pat. Nos. 5,665,344 and 6,190,652 there is disclosed a composition for attracting insects which comprises at least two volatiles of the Japanese Honeysuckle flower, one of said volatiles comprising cis-jasmone and the other being selected from linalool and phenylacetaldehyde. Beerwinkle et al. (1996) also took this approach with a five component mimic of Gaura species, and this work forms the basis of U.S. Pat. No. 6,074,634 to Lopez et al. In this patent there is disclosed a composition which includes a mixture of phenylacetaldehyde, methyl-2-methoxybenzoate, methyl salicylate and, optionally, 2-phenylethanol and/or limonene.

However, the problem with this approach is that the chemicals the moths are responding to may not be the most prominent ones in the profile. Indeed some components of these mixtures may even be insect deterrents. Evidence from coupled GC-MS-EAG studies (Plepys 2000, J. A. Pickett pers. comm. 2000) suggests that the components which produce the strongest EAG responses are often only very small peaks on a GC trace. Another problem with the mimic approach is learned behaviour. There is ample evidence that learning plays a major role in the foraging behaviour of many insects including noctuid moths. For H. armigera, Cunningham et al. (1998) have provided evidence that learning may be important. This is significant because attract-and-kill might fail in the field if the moths being targeted have already learned to forage on plants with different volatile profiles to the one being used as a model for the blend. Accordingly, there remains a need for an effective means of identifying attractants based on plant volatiles, which selects compounds which are genuinely attractive to the moth species in the field.

### SUMMARY OF THE INVENTION

According to one aspect of the present invention there is provided a method of selecting the components of a blend attractive to noctuid moths, comprising the steps of:
(1) measuring the attractiveness of a plurality of single candidate compounds; and
(2) selecting for blending those candidate compounds which show statistically significant attraction.

According to another aspect of the present invention there is provided a method of preparing a blend attractive to noctuid moths, comprising the steps of:
(1) measuring the attractiveness of a plurality of single candidate compounds;
(2) selecting for blending those candidate compounds which show statistically significant attraction; and
(3) blending two or more of the selected compounds.

Typically, the candidate compounds are selected from those present in extracts from plants which are attractive to insects, and generally from extracts from the leaves or flowers. The candidate compounds will generally be derived from different plants, and certainly need not be found together in the same plant in nature.

According to a further aspect of the present invention there is provided a composition for attracting noctuid moths when prepared from compounds identified by the above method or through using the method described above for preparing a blend.

Advantageously, the attractiveness of the single compounds is measured in an olfactometer. A typical olfactometer is illustrated in FIG. 1 and described below in more detail with reference to that Figure. Design of a suitable olfactometer is within the capability of the person skilled in the art, as such devices are known per se.

Typically the compositions of the invention are 3-5 component blends which are significantly attractive in the olfactometer to both sexes for H. armigera moths and other noctuid moths. In particular, such compositions are more attractive than the single chemicals, and more attractive than those blends in which the volatile components of a particular species are partially mimicked. Typically in the compositions of the invention 40-45% of the moths enter the test chamber of the olfactometer when the compositions of the invention are used, which is comparable with the best crop and weed plants.

It will be appreciated that the methodology of the invention may be employed to identify volatile plant attractants for a range of species, and to adapt these compositions to suit the learned foraging behaviour of natural populations of the insects.

Accordingly, in a further aspect of the invention there is provided a method of overcoming learned foraging behaviour in natural populations of moths, comprising the steps of:
(1) measuring the attractiveness of a plurality of single candidate compounds at a first time;
(2) selecting for blending those candidate compounds which show statistically significant attraction;
(3) measuring the attractiveness of a plurality of single candidate compounds at a second time later than said first time;
(4) selecting for blending those candidate compounds which show statistically significant attraction at said second time; and
(5) substituting those candidate compounds identified in step (4) for those identified in step (2) in attractant compositions.

Through use of the methodology of the invention, preferred compositions for attracting moth pests have been identified.

According to a still further aspect of the present invention there is provided a composition attractive to noctuid moths, comprising one or more floral volatiles in admixture with one or more leaf volatiles, the composition exhibiting a statistically significant level of attraction for noctuid moths in an olfactometer, with the proviso that the composition does not comprise phenylacetaldehyde, methyl-2-methoxybenzoate and methyl salicylate.

As used herein the term “floral volatile” refers to a volatile compound isolated from the flower of a plant, and which has a statistically significant level of attraction to moth pests as measured in an olfactometer.

In particular, the floral volatiles are benzene derivatives substituted by oxygen-containing groups such as the hydroxyl group, —ROH and —RCHO, where R is alkylene. Examples of floral volatiles are phenylacetaldehyde, 2-phenylethanol, benzyl alcohol. The lilac aldehydes, typically tetrahydrofuranyl derivatives of acetaldehyde, form another group of floral volatiles. A typical lilac aldehyde is 5-ethenyl-tetrahydro-α, 5-dimethyl-2-furanacetaldehyde.

As used herein, the term “leaf volatile” refers to a compound isolated from the leaf of a plant, and which has a statistically significant attraction to moth pests as measure in an olfactometer.

Typically the leaf volatiles are terpenoids, where that term is used to encompass not only terpenes of empirical formula C₁₀H₁₆, but also sesquiterpenes of formula C₁₅H₂₄, diterpenes of formula C₂₀H₃₂ and higher polymers, as well as various oxygen-containing compounds derived from terpene hydrocarbons such as their alcohols, ketones and camphors. Particular examples of such compounds are geraniol, 3-carene, methyl eugenol and limonene. In addition, the leaf volatiles may be other compounds, hereinafter referred to as “green leaf volatiles” where it is necessary to distinguish them from the terpenoid compounds discussed above, and these are generally 6-carbon alcohols, esters or aldehydes, for example, Z-3-hexenyl acetate or Z-3-hexenyl salicylate.

It will be appreciated that the person skilled in the art using the methodology described above may readily identify compounds which have a statistically significant attraction in the olfactometer, and can identify these either as floral volatiles or as leaf volatiles depending upon the source of the organic material from which the compound is isolated. It will nevertheless be appreciated that some compounds may be found in both floral tissue and leaf tissue, and may also be isolated from alternative sources. In addition, many structurally diverse compounds may be classified as or one or other of floral volatiles and leaf volatiles according to the definitions above, and all such compounds are envisaged.

Particularly preferred compositions comprise 0.1 to 10% of floral volatile in admixture with 0.1-15% leaf volatiles and 75-99.8% of an inert carrier.

Advantageously, the composition comprises 0.1-10% of a floral volatile selected from the group consisting of phenylacetaldehyde, 2-phenylethanol, benzyl alcohol and any one or more of the stereoisomers of 5-ethenyl-tetrahydro-α, 5-dimethyl-2-furanacetaldehyde, or an admixture thereof, and 0.1-15% of a leaf volatile selected from the group consisting of geraniol, 3-carene, methyl eugenol, or mixtures thereof, and/or a green leaf volatile selected from the group consisting of Z-3-hexenyl acetate and Z-3-hexenyl salicylate, or mixtures thereof.

The composition may include other compounds which do not display a statistically significant attraction to noctuid moths in the olfactometer. For example, the compositions may include α-pinene, cineole, γ-terpinene, linalool and/or methyl-1-butanol, as well as a variety of other compounds attractive to noctuid moths to a greater or lesser degree. In addition, blends which partially mimic the olfactory profile of specific plants may be included. In particular, a mixture of 1.4% α-pinene+1% cineole+0.4% limonene, hereinafter referred to as the “F3” blend which mimics the dominant terpenoids in A. floribunda may be included but this is, in any event, a source of limonene.

Particularly preferred compositions in accordance with this embodiment of the invention have the following composition:
PF3Hs=(1% phenylacetaldehyde+2.8% F3 blend+2% Z-3-hexenyl salicylate)
PF1=(1% phenylacetaldehyde+1.7% F3 blend+0.3% gamma-terpinene)
2 PF3Hs=(2% 2-phenylethanol+2.8% F3 blend+2% Z-3-hexenyl salicylate)
2 PF3=(2% 2-phenylethanol+2.8% F3 blend)
PBE3=(2% phenylacetaldehyde+2% benzyl alcohol+2% methyl eugenol)
PF32P=(1% phenylacetaldehyde+2.8% F3 blend+2% 2-phenylethanol)
PBE2=(0.5% phenylacetaldehyde+4% benzyl alcohol+2% methyl eugenol)
PBE1=(2% phenylacetaldehyde+2% benzyl alcohol+2% methyl eugenol)*
PF3=(1% phenylacetaldehyde+2.8% F3 blend)
PBEL=(PBE3+2% limonene)
PB2PS=(1% phenylacetaldehyde+2% benzyl alcohol+2% 2-phenylethanol+2% Z-3-hexenyl salicylate)
PBELo=(PBE3+2% linalool)
PBELa=(PBE3+2% 5-ethenyl-tetrahydro-α, 5-dimethyl-2-furanacetaldehyde)
PBEMb=(PBE3+2% methyl-1-butanol)
PBEHs=(PBE3+2% Z-3-hexenyl salicylate)

In addition, the invention provides the following compositions:
PB2P=(1% phenylacetaldehyde+2% benzyl alcohol+2% 2-phenylethanol)
P2P=(1% phenylacetaldehyde+2% 2-phenylethanol)
F3Ha=(2.8% F3 blend+2% Z-3-hexenyl acetate)
F3Hs=(2.8% F3 blend+2% Z-3-hexenyl salicylate)
since such compositions also show statistically significant attraction despite containing only one or other of compounds selected from floral volatiles and leaf volatiles. Still further examples:
PBE 4=0.5% phenylacetaldehyde, 0.5% benzyl alcohol, 0.5% methyl eugenol
PBE 5=0.2% phenylacetaldehyde, 0.2% benzyl alcohol, 0.2% methyl eugenol
PBES=2% phenylacetaldehyde, 2% benzyl alcohol, 2% methyl eugenol, 2% Z-3-hexenyl salicylate
PB2PS=1% phenylacetaldehyde, 2% benzyl alcohol, 2% 2-phenylethanol, 2% Z-3-hexenyl salicylate
PN1=1% phenylacetaldehyde, 2% benzyl alcohol, 2% linalool, 1% Z-3-hexenol, 2% eugenol, 1% benzaldehyde, 5% 3-hydroxy-benzaldehyde
PN4=1% phenylacetaldehyde, 2% benzyl alcohol, 2% linalool, 1% Z-3-hexenol, 2% eugenol, 1% benzaldehyde, 5% 3-hydroxy-benzaldehyde, 2% (−)-trans-caryophyllene
PBEu=2% phenylacetaldehyde, 2% benzyl alcohol, 2% eugenol
PBBE=2% phenylacetaldehyde, 2% benzyl alchol, 2% benzaldehyde, 2% methyl eugenol
PBBEu=2% phenylacetaldehyde, 1% Benzyl alcohol, 1% benzaldehyde, 2% eugenol
2 PF3Hs=2% 2-phenylethanol, 1.4% a-pinene, 0.4% limonene, 1% cineole, 2% Z-3-hexenyl salicylate

Although the compositions of the invention may also include compounds found not to have a statistically significant attraction, the specific combination of phenylacetaldehyde, methyl-2-methoxybenzoate, and methyl salicylate is not within the scope of the present invention. Likewise, a combination of phenylacetaldehyde, methyl-2-methoxybenzoate, methyl salicylate and 2-phenylethanol or limonene, or both, is excluded.

The compositions of the present invention typically include an inert carrier. Volatile compounds such as those of the invention may be formulated in a variety of inert carriers, the nature of which would be recognised by the person skilled in the art. They may be formulated in liquid or solid form, where appropriate, in a manner well understood by the person skilled in the art. Suitable liquid carriers include but are not limited to polyols, esters, methylene chloride, alcohol (such as C₁-C₄alcohol), vegetable oil or SIRENE base, although vegetable oils and SIRENE base are preferred. Suitable vegetable oils include olive oil, sesame oil, peanut oil, canola oil, cottonseed oil, corn oil, soybean oil, mineral oil, as well as methylated forms of these oils, or mixtures thereof, although canola oil is preferred. Aromatic and linear hydrocarbon solvents may also be included. The active ingredient mixture may also be incorporated in a solid substrate, such as clays, diatomaceous earth, silica, polyvinyl chloride, polystyrene, polyurethanes, ureaformaldehyde condensates, and starches. Other useful solid support matrices include expanded vermiculite and paraffinic or bees wax. SIRENE is an attract-and-kill formulation in paste form which uses pheromones and includes Permethrin to kill the male insects attracted by the pheromones when they come into contact with the paste, and for which IPM Technologies Ltd have worldwide marketing and development rights.

Mixtures of carriers are envisaged in the present invention and, for example, an aqueous/oil mixture in which the plant volatiles are dissolved in a miscible vegetable oil for subsequent admixture with a 10% solution of sucrose in water (sucrose being included as a feeding stimulant) are envisaged. Additionally, a small quantity of glycerol may be added to such a formulation as a humectant and a small quantity of polyvinyl alcohol added to form a skin over the droplets, with the aim of slowing desiccation.

Surprisingly, it has been found that SIRENE base (from which the pheromones and Permethrin are absent but which is generally referred to hereinafter as SIRENE) diluted with a miscible vegetable oil is a useful ingestible formulation. In this preferred formulation, the plant volatiles are included in the vegetable oil prior to admixture in this embodiment, and finally sieved icing sugar is added at 10% w/w as a feeding stimulant which, it should be noted, does not dissolve but becomes finely spread throughout the mixture. However, it will be appreciated that this novel vehicle has a more general utility, and this is described below.

As alluded to above in describing the preferred ingestible formulations, such formulations may include a variety of optional components or adjuvants, including but not limited to feeding stimulants, food sources, insect toxicants and other insect attractants such as insect pheromones. Yet other components which may be included in the formulation include humectants, preservatives, thickeners, antimicrobial agents, antioxidants, emulsifiers, film forming polymers and mixtures thereof. Additives which retard or slow the volatilization of the active mixture are also envisaged. Humectants may include polyols, sugar fractions (such as molasses), glycols and hygroscopic salts. Antioxidants which protect the vegetable oils and reduce polymerization of phenyl acetaldehyde are preferred. Film forming polymers include gum rosin, latex, polyvinyl pyrrolidone, polyvinyl alcohol, polyvinyl chloride, polyethylene, polyvinyl acetate and mixtures thereof. Additional optional additives include shellac, methyl methacrylate, and mixtures thereof.

In a preferred embodiment feeding stimulants for the adult insects or moths are included in the attractant composition and function to induce the target insets to contact and/or ingest the bait, particularly when formulated with an insecticide to effect control. Without being limited thereto, feeding stimulants such as fructose, fucose, glucose, and particularly sucrose, are preferred.

According to a further aspect of the present invention there is provided a base for an ingestible formulation comprising SIRENE base diluted with a miscible vegetable oil until it reaches a sufficiently low viscosity to be ingested by moth pests.

Typically the base is an inert carrier for physiologically active compounds, particularly those active in insects, and more particularly insect attractants or insecticides.

Advantageously said inert carrier includes a feeding stimulant.

Typically an insect attractant is formulated therewith. This maybe an insect attractant of the present invention or a conventional insect attractant such as a pheromone or a plant volatile composition of the type designed to mimic the olfactory components of a plant.

The compositions described above could be used in conjunction with trap crops to lure moth pests to the area of the trap crop where they might remain, and where the females might oviposit. The trap crop is then destroyed. They could also be used in traps for moth pests.

According to a still further aspect of the present invention there is provided a method of attracting moth pests to a locus, comprising the step of applying an attractant composition as described above to said locus.

The locus may be a trap crop, wherein the method comprises locating the attractant composition within or adjacent the trap crop. Alternatively, the locus may be a trap for a moth pest, wherein the method comprises applying the attractant composition to the trap, such as by locating an amount of the composition within a depot in the trap.

The attractant composition may be formulated in a manner known per se for spraying, as would be well understood by the person skilled in the art, and this is a convenient means for applying the composition to a trap crop. The components of the composition may also be applied separately or released by an attractant disseminator if desired.

Insect toxicants may also be included in the formulations of the invention.

Typically the toxicant is a pyrethroid or a carbamate. Preferred insect intoxicants include bifenthrin, carbaryl, methomyl, acephate, thiodicarb, cyfluthrin, malathion, chlorpyrifos, emamectin benzoate, abamectin, spinosad, endosulfan, and mixtures thereof. Bacterial and viral pathogens may also be included, as well as insect growth regulators or compounds eliciting behavior modification or disrupting physiological functions. These may include, for instance, pigments and/or dyes which may mark, attract, modify various insect behaviors, or which may be toxic. Combination of the insecticide with the attractant composition of this invention allows the use of significantly lower concentrations of insecticides to kill the adults under field conditions than would be used to control the insect pests with a normal commercial broadcast application of the same insecticides.

The attractant compositions may be used in a number of ways, including monitoring or controlling insect populations. In one preferred embodiment, the compositions may be placed within traps to monitor population changes. Precise monitoring will enable growers to reduce the number of insecticide applications when populations are low. In other embodiments, the attractants may be used to control pest populations by employing large numbers of traps (trap-out strategy).

It is envisioned that the attractants may be used in conjunction with any type of appropriate trap or attractant disseminator as known in the art. The attractant can be applied or disseminated using a variety of convention techniques, such as in an exposed solution, impregnated into a wicking material or other substrate, or incorporated in a deodorant dispenser. Further, the components of the attractant may be combined in a single dispenser provided within a single trap, or provided separately in a plurality of dispensers, all within a single trap. The attractant can be applied to the device undiluted, or formulated in an inert carrier. Volatilization can be controlled or retarded by inclusion of components as described above. Controlled, slow release over an extended period of time may also be effected by placement within vials covered with a permeable septum or cap, by encapsulation using conventional techniques, or absorption into a porous substrate.

One of ordinary skill will appreciate that the rate of release of the active ingredient mixture of the present invention may be varied by manipulation of the size of the reservoir and permeability of the matrix. The support or other delivery mechanisms of the present invention preferably provides release or volatilization of the active ingredient mixture of the invention for at least one week.

Application scenarios and methods of using the attractant composition of the present invention also include separate application of a feeding stimulant (such as molasses or sucrose solutions), combined with an insecticide, to plants by known methods, with the placement of the attractant composition in a manner which will attract moth pests to the feeding stimulant-insecticide mixture. Placement may include location in a strip in the same field which is upwind of the strip of the feeding stimulant-insecticide mixture. Another placement may involve a small area treated with the attractant composition in the centre of a larger area treated with the feeding stimulant-insecticide mixture. The attractant composition of the present invention may be applied in or on granules, plastic dispensers or wicks, for example, and may be applied parallel to sprays of a feeding stimulant-insecticide mixture. Cross-wind application may offer greater control of the insect population because of an increase in the area with effective volatile concentrations, and the foraging and ovipositing behavior in which the moths fly upwind within the plant canopy. Single point application of the attractant composition may also be used effectively, depending on the existing wind conditions. Plants which may be protected from insect pests include but are not limited to agronomically important crops such as cotton field corn, field peas, lupins, chick peas, sunflowers, sorghum, soybeans and vegetables, including seed corn, sweet corn, Cole crops, melons, beans and tomatoes.

In the practice of any of the above-described embodiment, an attractant is used as a trap bait or is otherwise applied to the locus of or in the vicinity of infestation in an amount effective to attract the target insect. Factors such as population density, precipitation, temperature, wind velocity, and release rate will influence the actual number of insects trapped.

The attractiveness of certain of the compounds discussed above to insects, and more particularly to noctuid moths, has not previously been recognised.

Accordingly, in a further aspect of the present invention there is provided the use of Z-3-hexenyl salicylate and methyl eugenol as attractants for noctuid moths. In particular, the invention provides the use of Z-3-hexenyl salicylate in this role, as its attractiveness to insects of any sort has not been recognised previously.

According to a still further aspect of the present invention there is provided an attractant composition comprising Z-3-hexenyl salicylate and/or methyl eugenol and an inert carrier.

Throughout this specification and the claims, the words “comprise”, “comprises” and “comprising” are used in a non-exclusive sense, except where the context requires otherwise.

It will be clearly understood that, although a number of prior art publications are referred to herein, this reference does not constitute an admission that any of these documents forms part of the common general knowledge in the art, in Australia or in any other country.

### BRIEF DESCRIPTION OF THE DRAWINGS

Preferred embodiments of the present invention will now be described by way of example only, with reference to the accompanying drawings in which:

FIG. 1 shows an olfactometer which may be used in selecting compounds suitable for use in the present invention;

FIG. 2 shows the attractiveness of plants in the olfactometer. The dotted line represents the minimum percentage of moths entering the test chamber required to give statistically significant attraction. The dashed bar (position 6) represents the published attractiveness of Gaura suffulta (Beerwinkle et al. 1996) which is used as a target standard of attractiveness. The pale bar is a blank olfactometer, with nothing in either chamber. Plants (from left to right) are:

1=Eucalyptus nova-anglica (New England peppermint), 2=Angophora floribunda (rough-barked apple), 3=Eucalyptus caliginosa (narrow-leafed stringybark), 4=Eucalyptus melliodora (yellow box), 5=Araujia hortorum (moth vine), 6=Gaura suffulta, 7=Calendula sp. (marigolds), 8=Chicorium intybus (chicory), 9=Helianthus annuus (sunflower), 10=Medicago sativa (lucerne), 11=Gaura lindheimerii, 12=Eucalyptus viminalis (white gum) 13=Zea mays (corn), 14=Bidens pilosa (cobbler's peg), 15=Cicer arietinum (chickpeas), 16=Linum usitatissimum (linseed), 17=Westringia fruticosa (white flowered Westringia), 18=Eremophila gilesii (Charleville turkey bush), 19=Galinsoga parviflora (yellow weed), 20=Hirshfeldia incana (Buchan weed), 21=Ixiolaena brevicompta, 22=Angophora floribunda (leaves only), 23=Sorghum vulgare (sorghum), 24=Nicotiana velutinum (wild tobacco), 25=Rhodanthe floribunda (large white sunray), 26=Malus domestica (apple blossom), 27=Echium plantagineum (Paterson's Curse), 28=Sonchus oleraceus (sowthistle) 29=Acacia sp.2, 30=Gossypium hirsutum (cotton), 31=Oenothera stricta (evening primrose), 32=Eremophila sturtii, 33=Cajanus cajan (pigeonpeas), 34=Dolichos lablab (Koala Lablab), 35=Acacia subulata, 36=Brassica napus (canola, 37=Westringia fruticosa (blue-flowered Westringia), 38=Jasminum officinale (jasmine), 39=blank olfactometer, 40=Lonicera japonica (Japanese honeysuckle), 41=Acacia cambadgeii (gidgee);

FIG. 3 shows the attractiveness of leaves and flowers of two plants in the olfactometer. Angophora floribunda (left), and Eucalyptus nova-anglica (right);

FIG. 4 shows the attractiveness of crude extracts in the olfactometer;

FIG. 5 shows the attractiveness of single chemicals in the olfactometer. The dotted line represents the minimum percentage of moths entering the test chamber required to give statistically significant attraction. Chemicals (from left to right) are:

1=Z-3-Hexenyl salicylate, 2=Phenylacetaldehyde, 3=2-Phenylethanol, 4=Benzyl alcohol, 5=Z-3-Hexenyl acetate, 6=3 carene, 7=Geraniol, 8=Limonene, 9=Methyl eugenol, 10=Benzyl benzoate, 11=Hexenyl benzoate, 12=Methyl-2-methoxybenzoate, 13=Cineole, 14=α-caryophyllene, 15=3-methyl-1-butanol, 16=Z-6-Nonenol, 17=Hexanol, 18=α-pinene, 19=Linalool, 20=eugenol, 21=Benzyl salicylate, 22=Benzaldehyde, 23 (pale bar)=Blank olfactometer, 24=Methyl salicylate, 25=Ethyl acetate, 26=β-caryophyllene, 27=Z-3 hexenol, 28=E-2-hexenal, 29=Z-3-hexenyl butyrate, 30=2-Hydroxybenzaldehyde, 31=α-pinene (R enantiomer);

FIG. 6 graphically illustrates the test percentages in the olfactometer of three volatiles plotted against concentration. Volatiles are formulated in Sirene. The line represents the minimum test % required for statistical significance;

FIG. 7 shows the attractiveness of chemical blends in the olfactometer. The dotted line represents the minimum percentage of moths entering the test chamber required to give statistically significant attraction. The blends (from left to right) are:
1=PF3Hs (1% phenylacetaldehyde+2.8% F3 blend+2% Z-3-hexenyl salicylate)
2=PF1 (1% phenylacetaldehyde+1.7% F3 blend+0.3% g-terpinene)
3=2 PF3Hs (2% 2-phenylethanol+2.8% F3 blend+2% Z-3-hexenyl salicylate)
4=2 PF3 (2% 2-phenylethanol+2.8% F3 blend) 5=PBE3 (2% phenylacetaldehyde+2% benzyl alcohol+2% methyl eugenol)
6=PF32P (1% phenylacetaldehyde+2.8% F3 blend+2% 2-phenylethanol)
7=PBE2 (0.5% phenylacetaldehyde+4% benzyl alcohol+2% methyl eugenol)
8=PBE1 (2% phenylacetaldehyde+2% benzyl alcohol+2% methyl eugenol)*
9=PF3 (1% phenylacetaldehyde+2.8% F3 blend)
10=PB2P (1% phenylacetaldehyde+2% benzyl alcohol+2% 2-phenylethanol
11=PBEL (PBE3+2% limonene)
12=PBELa (PBE3+2% 5-ethenyl-tetrahydro-α, 5-dimethyl-2-furanacetaldehyde)
13=P2P (1% phenylacetaldehyde+2% 2-phenylethanol)
14=PB2PS (1% phenylacetaldehyde+2% benzyl alcohol+2% 2-phenylethanol+2% Z-3-hexenyl sal.
15=PBE/FA/Z11-16OH (PBE3+fatty acid/Z-11-16OH mixture intended to mimic male hair pencils)
16=PBELo (PBE3+2% linalool)
17=PBEMb (PBE3+2% methyl-1-butanol)
18=PBEHs (PBE3+2% Z-3-hexenyl salicylate)
19=F3Ha (2.8% F3 blend+2% hexenyl acetate)
20=F3Hs (2.8% F3 blend+2% Z-3-hexenyl salicylate)
21=F3 (1.4% a-pinene+1% cineole+0.4% limonene)
22=Lopez blend (diagonal bars) (1.89% phenylacetaldehyde+0.73% methyl salicylate+1.96% limonene+1.83% 2-phenylethanol+1.59% methyl-23-methoxybenzoate)
23=Chin Blend B (3.5% eugenol+2.7% 2-phenylethanol+1.3% benzyl alcohol+3% benzaldehyde
24=Chin Blend A (5% phenylacetaldehyde+2% eugenol+1.7% 2-phenyethanol+1% benzyl alcohol+3% benzaldehyde
25=Blank (pale bars; olfactometer with no attractant)
26=Chin Blend C (4% 2-hydroxybenzaldehyde, 1.8% eugenol+0.8% benzyl alcohol+0.2% benzaldehyde+1.4% 2-phenylethanol;

FIG. 8 shows the effects of adding complexity to blends in the olfactometer, either within or across the classes of floral volatiles, and leaf volatiles. Light grey represents leaf volatiles and black is floral volatiles; and

FIG. 9 shows the changes in concentration of 6 plant volatiles over time, when placed in a trap in the field; and

FIG. 10 shows the numbers of moths killed per 50 m of furrow over four successive nights after applying treatments. A=Mythimna convecta, B=Helicoverpa armigera. Pheromone trap catches of H. armigera in the adjacent corn are also shown; and

FIG. 11 shows the percentages of dead moths found in furrows relative to the treated row for unsprayed and sprayed treatments (PF3Hs, Lopez and no-volatiles combined). The arrow indicates the position of the treated row. U5 to U1 are upwind furrows, from furthest to nearest, and D5 to D1 are downwind furrows.

### MODELS FOR PERFORMING THE INVENTION

### EXAMPLE 1

### Analysis of Plant Volatiles

Olfactometer Studies

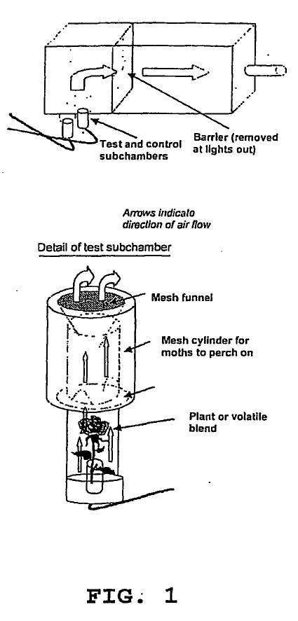

The design of our olfactometer (FIG. 1) has been described by Gregg et al. (1998) and is similar to that of Beerwinkle et al. (1996). The olfactometer is a two-choice system in which moths choose between two airstreams. One carries either a bouquet (about 200 g) of freshly cut plant stems with their bases in a flask of water, or plant volatiles formulated and presented as described below (the test chamber). The other carries clean air (the control chamber). Each run normally uses 50 moths, and runs are normally replicated three times for each sex and each attractant. Thus, olfactometer data points are usually each based on the responses of 150 moths. Moths are laboratory-reared, unmated and aged 1-4 (usually 2-3) days. We use two criteria for attractiveness, the test % (percentage of the total 150 moths which enter the test chamber), and success rate (number entering the test chamber relative to those entering the control chamber). The former is our primary criterion, and is the one presented here. Data are analysed using Zar's procedure for multiple comparison of proportions. With N=150, the minimum significant difference between two percentages using-this procedure is about 8%.

Analysis of Plant Volatiles

For collection of plant volatiles we use Solid Phase Micro Extraction (SPME) methods in the test airstream of the olfactometer, followed by thermal desorption from the SPME fibre. Analysis is by conventional GC-MS techniques on a Hewlett Packard 6890.

Analysis of volatiles from formulations, which we do to measure persistence, follows extraction from the base (Sirene or canola oil) for 1 h in hexane. A small quantity of supernatant is injected directly into the GC-MS. An internal standard, usually geraniol, is added to the hexane so that the amount of the test volatile can be quantified.

Formulations

For testing single chemicals in the olfactometer early in the project, we dissolve the synthetic chemical in canola oil at a concentration of 10%. 200 μl of this is absorbed into a cotton dental wick which is placed in the test airstream of the olfactometer. These lures are changed every 2 h during olfactometer runs. Later tests of single chemicals to determine the effects of varying concentration are done using Sirene formulations, as discussed below.

Plants in the Olfactometer

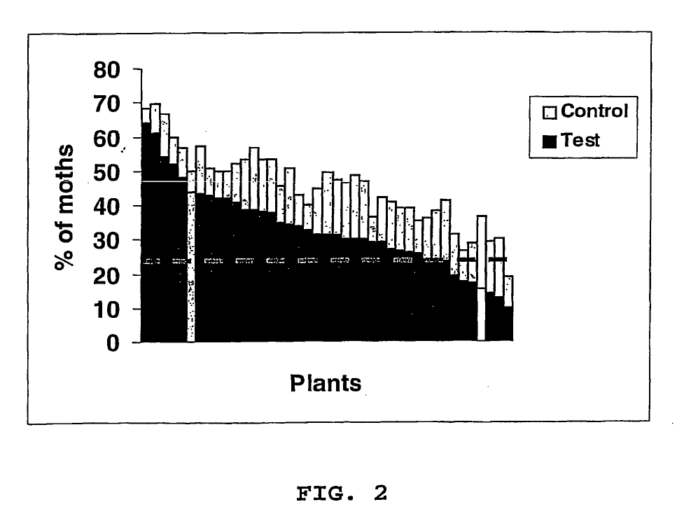

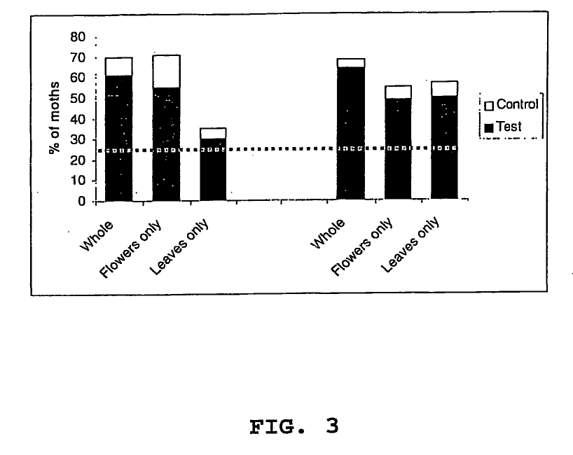

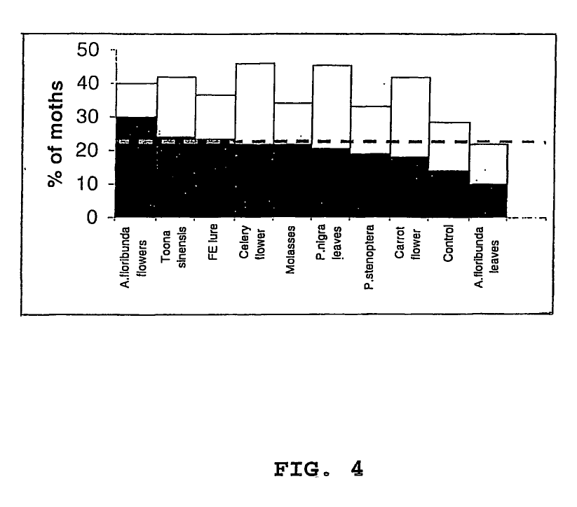

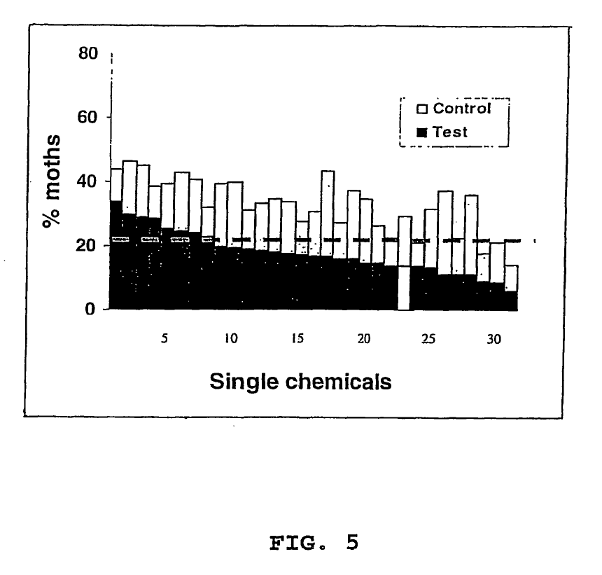

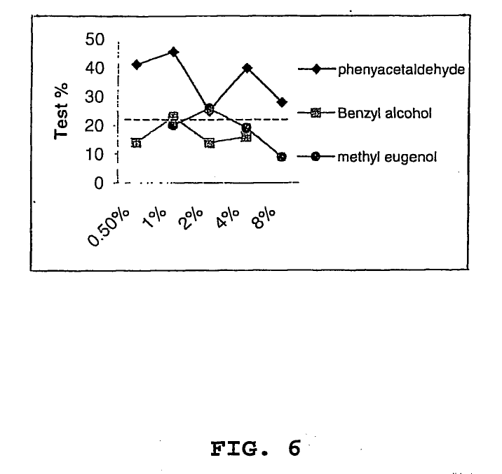

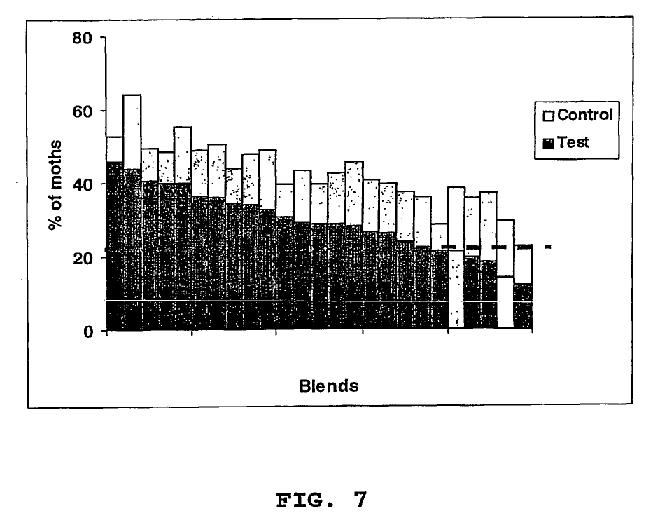

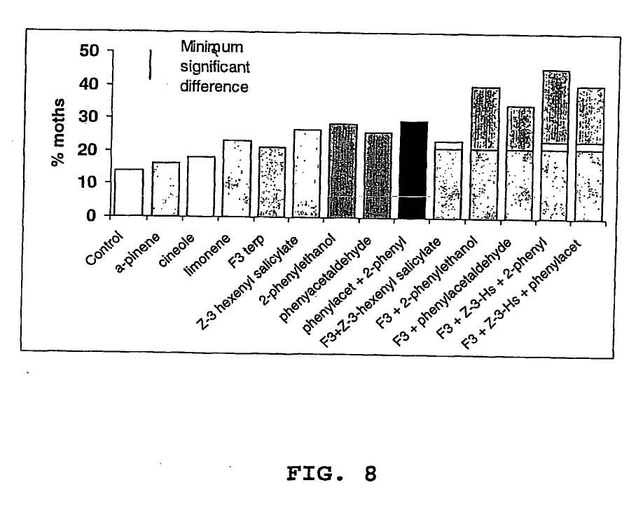

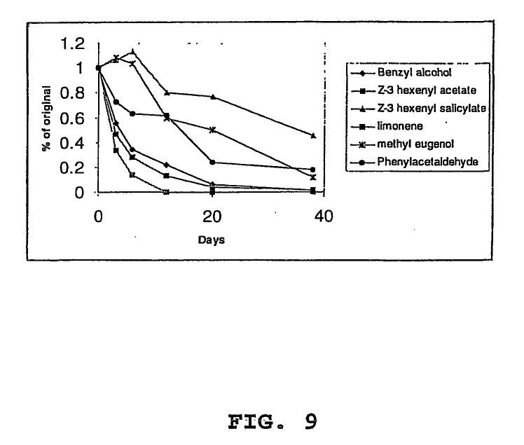

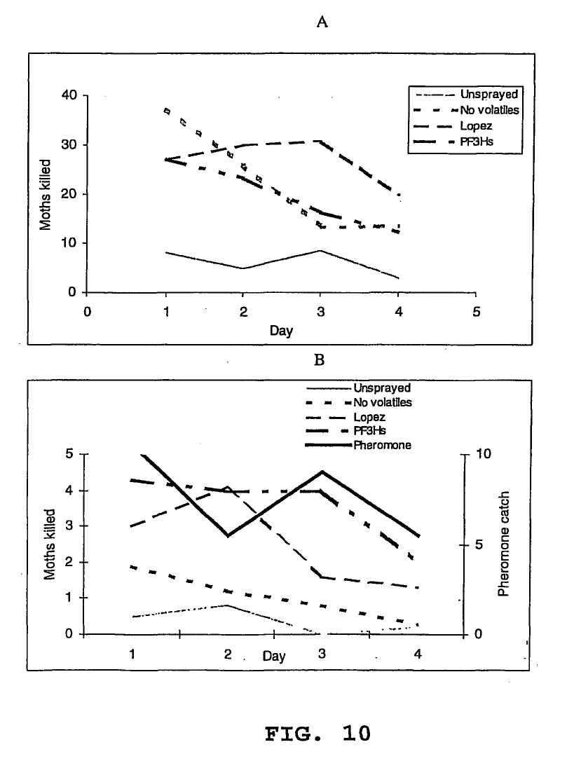

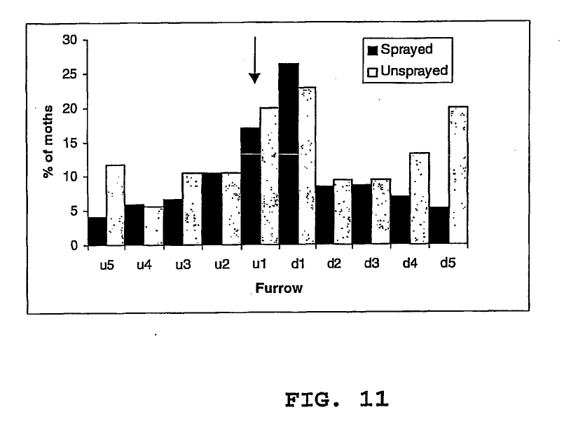

FIG. 2 summarises results with plants in the olfactometer. The data are for female moths which are the main target of this project—we also have data for male moths showing similar trends. Plants which were attractive to females were usually at least as attractive to males in the olfactometer. The dotted line in FIG. 2 represents the minimum test % level to give statistically significant difference from the control (blank) olfactometer test which is third from the right. On this criterion, all except 6 plants gave significant levels of attraction. There was significant variation in the attractiveness of the remaining plants. The dashed bar in FIG. 2 represents the published attractiveness of the best American Gaura species (G. suffulta, Beerwinkle et al. 1996), and was used as a target for our studies. Five species exceeded this attractiveness. Of these, four belong to the family Myrtaceae (gum trees, though one is actually an Angophora). The fifth, Araujia hortorum, is a weed belonging to the family Asclepidiaceae. None of these plants are suitable hosts for larval development of Helicoverpa spp.

After these came a group of plants almost as attractive as G. suffulta. They included Gaura lindheimerii. This group also included another eucalypt, E viminalis (white gum). It also included some crops or ornamental plants which are frequently observed to harbour large numbers of Helicoverpa moths, and which are suitable hosts for larval development. These included sunflowers, marigolds, maize and lucerne. These were followed by a long list of moderately attractive plants including many crops, weeds and Australian natives. This group included plants in the families Brassicacae and Fabaceae, including some legume crops known for their attractiveness for oviposition by Helicoverpa spp. and their suitability for larval development, such as chickpeas and pigeonpeas. Cotton was towards the end of this moderately attractive group. The six plants for which no significant attraction was recorded included two of the three Acacias we tested (and the third was only weakly attractive). They also included canola (a known larval host of Helicoverpa spp.) and the ornamental/weed species Japanese honeysuckle.

The particular attractiveness of Eucalyptus and Angophora in our studies may at first glance seem surprising in view of the unsuitability of these plants as larval hosts, but it is consistent with extensive observations of the pollen carried on the probosces of Helicoverpa spp. (Gregg 1993, Gregg and Del Socorro unpublished data). Even in circumstances where human observers find it difficult to locate flowering gum trees, Eucalyptus is often the dominant pollen type on the moths. Overall in our work, there was no correlation between the attractiveness of a plant in the olfactometer and its suitability as a larval host for Helicoverpa spp. We believe this is because our olfactometer measures attractiveness for adult foraging, not oviposition, in the unmated moths we used. The heirarchy of attractiveness might be different for mated moths.

For two of our most attractive plants, Angophora floribunda and Eucalyptus nova-anglica, we attempted to determine whether the attraction was in the leaves or the flowers. Bouquets of plant material containing leaves and flowers were compared with those containing only one or the other (FIG. 3). For each species, the highest levels of attraction were obtained from the combination of leaves and flowers, but significant levels of attraction were found to leaves and flowers alone. For A. floribunda the flowers were significantly more attractive than the leaves, but for E. nova-anglica there was no significant difference. We also did a comparison for E. nova-anglica between mature leaves and fresh young leaves, and no significant difference was found. These results suggest that there are attractive volatiles in both the leaves and the flowers, but the combination of these is more attractive than either alone.

Attractiveness of Crude Extracts

Crude extracts of various plant materials have been used to attract Helicoverpa spp. and a number of other noctuid moths for many years. These include molasses, known to be attractive to Helicoverpa zea since the 1930's (Ditman 1937), and a lure made from fermenting port wine and brown sugar which attracts armyworms (McDonald 1990). A final class of crude extracts are steam-distilled extracts of plants known to be attractive. In China wilted leaves of the trees Populus nigra and Pterocarya stenoptera are used to attract H. armigera moths, which are then killed by placing a plastic bag over the bunch of leaves. Crude extracts from these plants might also be attractive.

FIG. 4 shows the attractiveness of several of these mixtures. Though several showed significant or near significant levels of attraction, in no case were they as attractive as the best whole, fresh plants. Steam-distilled extracts of the flowers of A. floribunda were attractive, but leaf extracts were not. This mirrors the results obtained with fresh material (FIG. 3), but at lower levels of attraction. It suggests that some important volatiles are lost in the steam distillation process. Significant attraction was also obtained to a steam distilled extract from Chinese Toon tree, Toona sinensis. The armyworm fermentation (FE) lure was also significantly attractive, but field experience (Del Socorro unpublished data) suggest that it is much less attractive to moths in the subfamily Heliothinae than to the armyworms (Hadeninae), especially Mythimna convecta. The level of attraction to molasses was almost but not quite statistically significant. No significant attraction to the dried leaves of P. nigra or the steam distilled extract from P. stenoptera was found.

Volatile Analyses of Plants

We have volatile analyses, for almost all of the plants in FIG. 2. Typically, 10 to 20 peaks could be discerned in GC-MS traces for each plant, and most of these could be identified with reasonable confidence. In all about 120 chemicals have been identified from one or more plants. In many cases plants share many volatiles. Common chemical types included terpenoids (limonene, cineole, pinene etc), floral volatiles (phenylacetaldehyde, benzaldehyde etc), leaf alcohols and their esters (hexenol, benzyl alcohol and acetates or salicylates thereof), and nectar fermentation volatiles (alcohols and acetates).

We used these volatile profiles to construct a matrix (spreadsheet) in which the plants were arranged in order of attractiveness and the relative quantities of volatiles in each classified as +, ++, +++. This enabled us to identify the volatiles most commonly found in the most attractive plants.

Attractiveness of Single Chemicals

From the matrix described above we identified about 30 chemicals we considered worth testing in the olfactometer. Results are shown in FIG. 5. Eight chemicals gave statistically significant levels of attraction to females. In order of attractiveness these were: Z-3-hexenyl salicylate, phenylacetaldehyde, 2-phenylethanol, benzyl alcohol, Z-3-hexenyl acetate, 3-carene, geraniol and limonene. These results were obtained using canola oil formulations. Subsequently the attractiveness of phenylacetaldehyde, 2-phenylethanol, benzyl alcohol and Z-3-hexenyl salicylate was confirmed in Sirene formulations. The remaining 3 chemicals have not been studied in this formulation, but a ninth chemical, methyl eugenol, was also found to be attractive in Sirene.

However, in no case did the level of attraction with single chemicals approach that of the best whole plants. This suggests that moths may respond more to blends than to single chemicals. Also, we have only tested each chemical at one concentration. It is possible that some may be attractive at other concentrations. Some may even be repellant if the concentration is too high.

The attractiveness of single chemicals in the olfactometer is shown in FIG. 5. From this it will be appreciated that compounds 1 to 8 show statistically significant attractiveness to moth pests, and compound 9 is very close to being statistically significant. The remainder of the compounds tested were not statistically significantly attractive on their own.

Sirene Base Versus Canola Oil, and Single Chemical Concentration Studies

FIG. 6 shows the attractiveness in the olfactometer of three single chemicals at different concentrations. These results are not directly comparable with those in FIG. 5 because the chemicals were formulated in Sirene base rather than canola oil, and release rates from this formulation are much slower. In the case of phenylacetaldehyde and benzyl alcohol there appears to be an optimum concentration of about 1%, though the results for 2% phenylacetaldehyde are anomalous. For methyl eugenol the best result appeared to be about 2%. For this chemical significant levels of attraction were obtained in this system, whereas the attraction was not quite statistically significant when the formulation was in canola oil. In general, the results suggest that responses of moths in the olfactometer are not especially sensitive to concentration, but that they can decline if the concentration is too high. This means that caution must be used in concluding from the canola oil data (FIG. 5) that any particular chemical is not attractive. In view of the higher concentrations used, and the higher release rates from canola oil compared to Sirene, it may simply be that the moths were exposed to excessive concentrations.

These three volatiles where test results are shown in FIG. 6 were tested in different concentrations because they are components of one of the first blends we tested, PBE, which has been used as a standard for field trials. We have not done dose-response studies in the olfactometer for any other volatiles. We have investigated 2% concentrations in Sirene of Z-3-hexenyl salicylate (test %=26.7 compared with 33.8 in canola oil) and 2-phenylethanol (26.4% vs 29%). Both were significantly attractive in both formulations.

### EXAMPLE 2

### Attractiveness of Chemical Blends Formulations

For testing chemical blends in the olfactometer and in field trapping experiments, we are formulating in Sirene, a mixture of anti-oxidants and UV protectants which was developed by Novartis for attract-and-kill of several moth species using pheromones, and for which IPM Technologies Ltd have worldwide marketing and development rights. Chemicals are mixed into Sirene in the barrel of a syringe using a slow (4 rpm) broad-bladed propeller, which stirs the highly viscous material for 3 h. Formulations are then stored at 40 C in the sealed syringe for periods of up to 2 weeks. Sirene formulations are tested in the olfactometer by placing about 100 mg on a horizontal square (1×1 cm) of plastic (Corflute®), which is then placed on a pin in the test airstream of the olfactometer. For field testing, the plastic square carrying the formulation is held horizontally under the lid of the AgriSense trap, in the same position that a pheromone lure would be, using a screw.

Our second, ingestible, formulation is “sloppy Sirene” This consists of Sirene diluted with SprayTech® oil (a proprietary emulsifiable vegetable oil formulation produced by Agrobest Australia Pty. Ltd., or canola oil) to the point where it becomes runny enough to be ingested by moths. The plant volatiles are included in the oil. Finely sieved icing sugar is added at 10% w/w as the feeding stimulant. It does not dissolve but becomes finely spread throughout the mixture.

The approach then to prepare the blends was to combine chemicals which were attractive in single chemical tests, but which are not necessarily found together in the same plant in nature. We call these “super-blends”. We now have several 3 to 5 component blends which are significantly attractive in the olfactometer to both sexes of H. armigera moths, more attractive than the single chemicals, and more attractive than the patented blend of Lopez et al. (2000) (FIG. 7). Of the 25 blends we have tried, statistically significant levels of attraction have been obtained with 20. The best of these are giving test % levels of 40-45% in the olfactometer, which is comparable with the best of the crop and weed plants, though still considerably less than the best Myrtaceae.

The best blends always contain a floral volatile (phenylacetaldehyde or 2-phenylethanol) plus a leaf volatile(s), either the F3 blend, a mixture mimicking the 3 dominant terpenoids in A. floribunda, or methyl eugenol. Leaving out either of these component classes results in a decline in attractiveness, no matter what other components are added (including green leaf volatiles). Such a pattern is consistent with the requirement for both flowers and leaves for optimum attractiveness in the olfactometer.

Systematic Studies of Blend Complexity

In these studies (FIG. 8) we systematically investigated the effects of adding complexity to blends, either within the groups of leaf volatiles or flower volatiles, or by combining the two. The three dominant terpenoids in A. floribunda had only weak activity (light grey bars in FIG. 8). The only one which was statistically significant was limonene. When combined in the F3 blend, their attractiveness was about the same a limonene. Likewise, the two floral volatiles phenylacetaldehyde and 2-phenylethanol (black bars) are significantly attractive, but when combined they are not more attractive than either alone. On the other hand, combination across the classes (ie floral volatiles and terpenoids) does result in significantly increased attractiveness. The green leaf volatile Z-3-hexenyl salicylate is significantly attractive on its own, and when added to the terpenoid/floral volatile mix it may slightly increase attractiveness (though the difference is not statistically significant). We have not investigated the combination of multiple green leaf volatiles.

### EXAMPLE 3

### Persistance of Plant Volatiles

Formulations containing 6 plant volatiles in a mixture in Sirene were left in AgriSense traps under field conditions during summer at Armidale for several weeks. At intervals subsamples were removed and analysed for volatile content using the hexane extraction method described above. There were three replicate samples at each interval. This work was repeated on a similar formulation stored in a sealed 20 ml. syringe in a refrigerator.

FIG. 9 shows the concentration of these 6 volatiles, expressed as a percentage of the original. Some volatiles, such as benzyl alcohol, limonene and Z-3-hexenyl acetate, have half-lives of only a few days. Others, such as phenylacetaldehyde, methyl eugenol and Z-3-hexenyl salicylate, have half-lives of 15-40 days. It is clear that this differential loss is due to differences in volatility. No novel compounds suggestive of oxidative or other types of degradation were seen in the GC-MS traces. The implication of these results is that blended lures placed in the field will begin to vary from their original proportions almost immediately. If the proportions in the blend are crucial then it will remain attractive for only a short time (the limited evidence from our work, however, is that proportions are not especially critical). Another implication from this work is that persistence and release rates are negatively correlated, and even if different substances are in the same concentration in a blend, they will not be perceived as such by an insect.

We have also studied the half-lives of the same 6 volatiles in Sirene in a 20 ml syringe kept in refrigerator. The syringe was not gas-tight, but the tip was covered with a plastic cap. After 40 days, between 60-80% of the original amount of each volatile was still present. This result suggests that shelf life of volatile formulations would be good, given reasonable precautions to exclude air and keep temperatures low.

Finally, we studied the persistence in canola oil of three of these volatiles. There was a good correlation with persistence in Sirene, and half lives in canola oil were about one fifth of those in Sirene. This ratio is probably fairly representative of other volatiles, and for vegetable oil formulations such as Spraytech® oil, in general.

### EXAMPLE 4

### Field Tests and Toxicology Insecticides

For the ingestible plant volatile formulations we have tested the carbamates carbaryl and methomyl. The concentration of active ingredient in the final formulation has been 0.5 to 1%. We use technical grade insecticides. Insecticides are dissolved in ethanol before being added to the formulations.

Exemplary formulations are as follows:

A. Oil/Water Mixes

Aqueous Component

Water 100 ml
Sucrose 15 g
Glycerol 30 ml
10% polyvinyl alcohol in distilled water 20 ml
Brilliant green dye 10 mg
   Oil Components
   1. PBE Blend
200 μl phenylacetaldehyde
200 μl benzyl alcohol
200 μl methyl eugenol
200 μl sorbitan monostearate (emulsifying agent)
10 ml Spraytech® oil or canola oil
   2. Lopez Blend
208 μl phenylacetaldehyde
232 μl limonene
220 μl 2-phenylethanol
100 μl methyl salicyate
196 μl methyl-2-methoxybenzoate
200 μl sorbitan monostearate (emulsifying agent)
10 ml Spraytech® oil or canola oil
   3. PBELo Blend
200 μl phenylacetaldehyde
200 μl benzyl alcohol
200 μl methyl eugenol
200 μl linalool
200 μl sorbitan monostearate (emulsifying agent)
10 ml Spraytech® oil or canola oil
   Insecticides
1 g of technical carbaryl or methomyl
10 g analytical grade ethanol
Add 8 ml of aqueous component, 1 ml of oil component and 1 ml of insecticide component. Mix and seal in full vial. Shake before use.
   B. “Sloppy Sirene” Formulations
Volatile components as in the above blends.
5 g Sirene
3 g Spraytech® oil. or canola oil
1 g sieved icing sugar
1 g insecticide mixture.
20 mg Brilliant Green dye.
Stir and seal in full vial. Stir before use.
   Laboratory Feeding Studies

To determine whether moths will feed on the ingestible formulations, and whether they die as a result, we place a single moth in a 1 litre plastic container which has 50 μl of the formulation as a single droplet in a beer bottle cap, on the bottom of the container. In some experiments laboratory-reared moths are used in a constant 25° C. reverse-cycle system. In others natural cycle laboratory moths are used in containers placed outdoors. Moths are observed at regular intervals and dead ones removed for dissection. At the end of the scotophase all surviving moths are removed and after a further hour are dissected. On dissection under a binocular microscope, the presence of green dye in the digestive tract indicates that moths have ingested the material.

In these studies small droplets (50-100 μl) were placed in a 1 litre container with the moths, so they spent the night in close proximity (within 10 cm) of the droplet. Table 1 shows that in these conditions most moths ingested the droplet. Kill rates of around 70-100% were obtained, except in one trial when the formulation did not contain an insecticide, when no mortality occurred. Formulations with 1% methomyl killed all moths; for carbaryl it was 69-94%.

TABLE 1
Summary of laboratory feeding experiments.

| | | | | | | % containing | |
|---|---|---|---|---|---|---|---|
| | For- | | Lo- | Number, | % | dye | |
| Trial | mulation | Toxicant | cation | age & sex | killed | Dead | Alive |
| 1 | Aqueous | 1% | Lab | 16 males, | 68.8 | 100.0 | 0.0 |
| | | carbaryl | | 1-4d | | | |
| 1 | Aqueous | none | Lab | 16 males, | 0.0 | NA | 62.5 |
| | | | | 1-4d | | | |
| 2 | aqueous | 1% | Lab | 32 | 75.0 | 95.8 | 0.0 |
| | | carbaryl | | females | | | |
| | | | | 1-3d | | | |
| 3 | sloppy | 1% | Lab | 16 | 93.8 | 100.0 | 0.0 |
| | Sirene | carbaryl | | females | | | |
| | | | | 2-3d | | | |
| 4 | aqueous | 1% | Field | 15 | 93.3 | 71.4 | 0.0 |
| | | carbaryl | | females | | | |
| | | | | 2-3d | | | |
| 4 | sloppy | 1% | Field | 15 | 80.0 | 91.7 | 0.0 |
| | Sirene | carbaryl | | females | | | |
| | | | | 2-3d | | | |
| 5 | aqueous | 1% | Field | 12 | 100.0 | 0.0 | NA |
| | | methomyl | | females | | | |
| | | | | 3d | | | |
| 5 | sloppy | 1% | Field | 12 | 100.0 | 25.0 | NA |
| | Sirene | methomyl | | females | | | |
| | | | | 3d | | | |

Ingestion of the formulation, as indicated by the presence of green dye, was always lethal. No surviving moths (carbaryl only) were found to have internal dye. On the other hand, not all dead moths had ingested the material. Most of those which had not (as indicated by the absence of internal dye) carried external dye, on the legs, proboscis or tip of the abdomen. These moths were presumed to have contacted the lure either accidentally or when attempting to feed, and been killed by the contact activity of the insecticide. In the case of carbaryl, which has relatively weak contact activity, there were only a few such moths. In the case of methomyl, a much more contact-active carbamate, all the dead moths carried external dye, sometimes only in very small traces. There were very few with internal dye, and then only in small quantities. It is likely that these moths were killed by contact before they were able to ingest much of the formulation. 1% methomyl is probably an gross overkill; concentrations could be greatly reduced, which would improve safety for the applicator.

All surviving moths had not ingested the material. These moths, up to about 30% of the total, were mostly young. In the case of males in trial 1, combining both the insecticide and non-insecticide trial, 14/16 (87.5%) of moths aged 2 days and older ingested the material, while for moths aged one day it was 6/16 (37.5%). In trial 2, with females, the rates were 30% for 1 day old moths, and 100% for both 2 and 3 day old moths.

There are a number of encouraging findings from this work. One is that if moths can be retained in close proximity to the volatile formulations, they will eventually ingest them, even when their base consists entirely of such unpromising organic materials as Sirene, SprayTech® oil and canola oil. Another is that carbamate insecticides in relatively low concentrations will kill moths, both by stomach and contact activity. This is despite the likely existence of carbamate resistance in our lab moths, which were derived from the Darling Downs where such resistance is common. The third encouraging result is the persistence of “sloppy Sirene” formulations. Oil/water mixtures separate into two phases within a few hours, even when a highly miscible oil such as SprayTech® is used. Plant volatiles and the insecticides are in the oil phase, and the sucrose is in the water. Eventually the droplet resembles a miniature poached egg. The water phase dries and the oil becomes tacky or smeared and not ingestible. In contrast, “sloppy Sirene” formulations do not separate and retain their shape and liquidity all night, and possibly longer.

Field Wind Tunnels

These are used to observe behavioural responses of moths around lures, and to record kill rates of different blends and formulations, in semi-natural conditions. Clear polythene is stretched around a series of steel hoops 1.2 m in diameter which are suspended from a horizontal pipe Flywire is placed over each end. The wind tunnels are hung just above ground level and oriented parallel to the prevailing wind. Lures (3 bottle tops containing 100-200 μl of the attractant under test) are placed on a plastic dinner plate in the upwind end and 20-50 moths are released into the downwind end about 1 h before sunset. Lab reared female moths produced under natural photoperiod are used. Behavioural responses are observed using night vision glasses. In the morning both live and dead moths are collected and dissected to determine from the presence of green dye whether they have fed on the lure.

We do not routinely run control wind tunnels, that is, with no lure or toxicant in them to determine the level of mortality which may result from any stresses associated with containment inside the wind tunnel for the night. We have, however, done this on several occasions, with no mortality observed. However, experience has shown that kill rates vary with weather. On cool and/or still nights, low kills are common, and observations with night vision glasses reveal little moth activity, especially flight, on such nights. For this reason caution is required in comparing results obtained on different nights, and it is best to run several wind tunnels with different treatments on the same night.

Results of 22 trials involving 51 wind tunnel/nights are shown in Table 2. As well as comparing blends, we have used field wind tunnels to compare formulations and investigate possible roles of visual and gustatory stimuli, using artificial flowers and sucrose or molasses respectively.

TABLE 2
Field wind tunnel trials. Treatments with the same trial number
were conducted on the same night, and percentage kills for
those treatments, within the same trial, which are followed by
different letters are significantly different by Zar's multiple range test
for proportions, P < 0.05. Blend identifications are as for FIG. 7.

| | | | | | | |
|---|---|---|---|---|---|---|
| Trial | Location | Attractant | Toxicant | N moths | N killed | % killed |

Comparison of blends

| | | | | | | |
|---|---|---|---|---|---|---|
| 1 | Bowen | PBELo | 0.5% | 38 | 19 | 50.0 |
| | | | methomyl | | | |
| 2 | Bowen | PF3Hs | 0.5% | 44 | 29 | 65.9 |
| | | | methomyl | | | |
| 3 | Bowen | PF3Hs | 0.5% | 39 | 29 | 74.4 a |
| | | | methomyl | | | |
| 3 | Bowen | PBELo | 0.5% | 39 | 19 | 48.7 b |
| | | | methomyl | | | |
| 4 | Yanco | PBELo | 0.5% | 43 | 27 | 62.8 c |
| | | | methomyl | | | |
| 4 | Yanco | PBE | 0.5% | 30 | 11 | 36.7 b |
| | | | methomyl | | | |
| 4 | Yanco | 2PF3Hs | 0.5% | 43 | 3 | 7.0 a |
| | | | methomyl | | | |
| 4 | Yanco | PF3Hs | 0.5% | 43 | 11 | 25.6 b |
| | | | methomyl | | | |
| 5 | Yanco | PBELo | 0.5% | 45 | 16 | 35.6 a |
| | | | methomyl | | | |
| 5 | Yanco | No volatiles | 0.5% | 45 | 13 | 28.9 a |
| | | | methomyl | | | |
| 6 | Yanco | PBELo | 0.5% | 52 | 24 | 46.2 a |
| | | | methomyl | | | |
| 6 | Yanco | PF3Hs | 0.5% | 48 | 33 | 68.8 b |
| | | | methomyl | | | |
| 7 | Yanco | PBELo | 0.5% | 47 | 22 | 46.8 a |
| | | | methomyl | | | |
| 7 | Yanco | PF3Hs | 0.5% | 50 | 33 | 66.0 b |
| | | | methomyl | | | |
| 7 | Yanco | PF32P | 0.5% | 50 | 27 | 54.0 a |
| | | | methomyl | | | |
| 7 | Yanco | PF4 | 0.5% | 49 | 33 | 67.3 b |
| | | | methomyl | | | |
| 8 | Yanco | 8% Phenylacetaldehyde | 0.5% | 43 | 11 | 25.6 b |
| | | | methomyl | | | |
| 8 | Yanco | PF3Hs | 0.5% | 42 | 27 | 64.3 c |
| | | | methomyl | | | |
| 8 | Yanco | 2% Phenylacetaldehyde | 0.5% | 42 | 14 | 33.3 b |
| | | | methomyl | | | |
| 8 | Yanco | No volatiles | 0.5% | 45 | 5 | 11.1 a |
| | | | methomyl | | | |

Effects of varying attractant
concentration and formulation

| | | | | | | |
|---|---|---|---|---|---|---|
| 9 | Yanco | 8% PBELo | 0.5% | 49 | 14 | 28.6 b |
| | | | methomyl | | | |
| 9 | Yanco | 2% PBELo | 0.5% | 49 | 33 | 67.3 c |
| | | | methomyl | | | |
| 9 | Yanco | 0.5% PBELo | 0.5% | 47 | 25 | 53.2 c |
| | | | methomyl | | | |
| 9 | Yanco | No volatiles | 0.5% | 47 | 3 | 6.4 a |
| | | | methomyl | | | |
| 10 | Armidale | PBELo (aqeuous) | 1% | 65 | 7 | 10.8 a |
| | | | carbaryl | | | |
| 10 | Armidale | PBELo (sloppy Sirene) | 1% | 67 | 8 | 11.9 a |
| | | | carbaryl | | | |
| 11 | Armidale | PBELo (aqeuous) | 1% | 38 | 11 | 28.9 a |
| | | | methomyl | | | |
| 11 | Armidale | PBELo (sloppy Sirene) | 1% | 39 | 2 | 5.1 b |
| | | | methomyl | | | |

Blends presented with artificial flowers

| | | | | | | |
|---|---|---|---|---|---|---|
| 12 | Bonnington | PBELo + flowers | 0.5% | 33 | 21 | 63.6 |
| | | | methomyl | | | |
| 13 | Bonnington | PBELo + flowers | 0.5% | 23 | 8 | 34.8 a |
| | | | methomyl | | | |
| 13 | Bonnington | PBELo | 0.5% | 24 | 8 | 33.3 a |
| | | | methomyl | | | |
| 14 | Bonnington | PBELo + flowers | 0.5% | 32 | 12 | 37.5 a |
| | | | methomyl | | | |
| 14 | Bonnington | No volatiles + flowers | 0.5% | 33 | 13 | 39.5 a |
| | | | methomyl | | | |
| 15 | Bonnington | PBELo + flowers | 0.5% | 29 | 12 | 41.4 b |
| | | | methomyl | | | |
| 15 | Bonnington | No volatiles + flowers | 0.5% | 29 | 4 | 13.8 a |
| | | | methomyl | | | |
| 16 | Yanco | 6% PBELo | 0.5% | 35 | 17 | 48.6 b |
| | | | methomyl | | | |
| 16 | Yanco | No volatiles + flowers | 0.5% | 38 | 11 | 28.9 a |
| | | | methomyl | | | |
| 16 | Yanco | 6% PBELo + flowers | 0.5% | 37 | 16 | 43.2 b |
| | | | methomyl | | | |

Molasses vs no molasses

| | | | | | | |
|---|---|---|---|---|---|---|
| 17 | Bowen | PF3Hs | 0.5% | 39 | 9 | 23.1 a |
| | | | methomyl | | | |
| 17 | Bowen | PF3Hs + molasses | 0.5% | 38 | 19 | 50.0 b |
| | | | methomyl | | | |
| 17 | Bowen | PF3Hs + molasses | none | 37 | 6 | 16.2 a |
| 18 | Bowen | Molasses only | 0.5% | 34 | 5 | 14.7 a |
| | | | methomyl | | | |
| 18 | Bowen | PF3Hs | 0.5% | 35 | 10 | 28.6 b |
| | | | methomyl | | | |
| 18 | Bowen | PF3Hs + molasses | 0.5% | 34 | 15 | 44.4 c |
| | | | methomyl | | | |
| 19 | Bowen | Molasses only | 0.5% | 40 | 10 | 25.0 a |
| | | | methomyl | | | |
| 19 | Bowen | PF3Hs | 0.5% | 41 | 9 | 22.0 a |
| | | | methomyl | | | |
| 19 | Bowen | PF3Hs + molasses | 0.5% | 41 | 23 | 56.1 b |
| | | | methomyl | | | |
| 20 | Bowen | Molasses only | 0.5% | 37 | 5 | 13.5 a |
| | | | methomyl | | | |
| 20 | Bowen | PF3Hs | 0.5% | 38 | 17 | 44.7 b |
| | | | methomyl | | | |
| 20 | Bowen | no volatiles | 0.5% | 39 | 9 | 23.1 a |
| | | | methomyl | | | |

Plate soaked in 20% sucrose vs no sucrose

| | | | | | | |
|---|---|---|---|---|---|---|
| 21 | Bonnington | PBELo + sucrose | 0.5% | 20 | 12 | 60.0 |
| | | | methomyl | | | |
| 22 | Bonnington | PBELo | 0.5% | 28 | 22 | 78.6 a |
| | | | methomyl | | | |
| 22 | Bonnington | PBELo + sucrose | 0.5% | 30 | 26 | 86.7 a |
| | | | methomyl | | | |

Trials 1-8 compared different blends. All blends except for 2 PF3HS in trial 4 gave substantial kills. When formulations with no attractant volatiles were included (trials 5 and 8), kills were lower than all formulations where attractants were included, though the difference was only statistically significant in trial 8. PF3HS and PBELo were consistently effective, with the former giving significantly higher kills than the latter on three of four occasions when the two were included in the same trial. PF3Hs also gave significantly higher kills than phenylacetaldehyde alone, whether the latter was present in the same concentration as in PF3Hs (2%) or equivalent to the total concentration of all chemicals in PF3Hs (8%) (trial 8). Varying the concentration of the total blend of PBELo indicated an optimum concentration of 0.5-2%, which gave significantly higher kills than 8%. All blends in this trial gave significantly higher kills than the formulation without PBELo. Comparisons between the aqueous and sloppy Sirene formulations did not give clear results, probably because they were conducted on cool nights and kills were low. In trial 9, no significant difference was obtained, but in trial 10, the aqueous formulation gave a significantly higher kill than sloppy Sirene.

Observations with night vision glasses provided insights into why moths sometimes do not contact or ingest the lures. Many moths were observed to approach the dinner plate containing the lures. Characteristic plume-following and hovering flight behaviour, as seen around pheromone traps, made it obvious they were responding to the volatiles. Moths made repeated approaches to the lures, hovered and then backed off. This suggested that further close-range stimuli, perhaps visual, were required to induce landing and contact with the lure. This hypothesis was tested by placing the attractant in the centre of a plastic artificial flower, mimicking a gerbera. In trials 13 and 16, presenting PBELo in Sirene on these flowers did not significantly increase kill rates, compared to the standard method of presentation in bottle tops. This suggests that either visual stimuli are not important, or the plastic flower used was not an adequate stimulus. Adding PBELo to flowers produced different results in two trials. In trial 14 it did not significantly affect the kill, which was higher than normally associated with treatments without attractant chemicals (eg trials 8 and 9). However the result was not repeated in trials 15 and 16, where PBELo did significantly enhance the attractiveness of the artificial flowers. These results suggest that either visual stimuli are less important than olfactory ones, or the plastic artificial flower used was not an adequate visual stimulus.

Sometimes moths would land on the plate and walk around, probing the plate with their probosces as if seeking further stimuli, perhaps gustatory. Trials 17-20 therefore investigated the effects of placing molasses, containing 0.5% methomyl, on the plate surrounding the lures. In trials 17-19 the presence of molasses significantly enhanced the kills obtained with PF3Hs, even though molasses alone was not particularly attractive (trials 18-20), and molasses without insecticide did not cause much mortality (trial 17). In two further trials, 21 and 22, the lures (PBELo) were presented on paper plates which had been soaked in 20% sucrose and allowed to dry. On these, moths remained for longer and probed more extensively. This suggests that gustatory stimuli are important in retaining moths near an attractant, and inducing them to contact it. High levels of mortality were obtained in these two trials, though in trial 22 the kill was not significantly higher than that without the sucrose. However, combined with the results of the molasses trials, this suggests that placing volatile lures in the centre of larger areas treated with sucrose/insecticide or molasses/insecticide formulations might be effective,

Field Tests (Trapping)

For field tests of the attractiveness of different blends we use AgriSense pheromone traps of the canister type (Gregg and Wilson 1991). Traps are usually cleared three times weekly, and the catch sorted. Moths are sexed and the females dissected to determine mated status. We normally test 5 formulations at a time, replicated 4 times, so there are 4 rows of 5 traps, each row containing one replicate of each formulation. Traps are spaced 50 m apart and are rotated, within a row, between catch intervals. Experiments are analysed as Latin squares. Field trials at Cecil Plains were conducted in soybeans, cotton, chickpea, wheat, fallow land and sunflower, and at Kununurra and China, in cotton.

Table 3 summarises the results of all our field trials. We are field testing our blends in the Darling Downs during the summer months, and the Ord River and Bowen, Queensland during the winter. We also conducted field trials at Henan province in China in August 1999. Although the blends that were tested in the field were found to be highly attractive to both sexes in the olfactometer, the numbers caught in the field have been very low relative to those caught by pheromone traps in the same experiments. Typically, plant volatiles caught only about 5-10% of pheromone catches. The sex ratio of moths caught in plant volatile traps is highly variable. Often, catches from the plant volatiles tended to be mostly females. We are not sure whether this is because the volatiles are inherently more attractive to females, or because the adjacent pheromone traps (which catch only males), are “robbing” the volatile traps of males. On the other hand, there were a few cases where the catch was mostly male, notably during the sunflower experiment at Wamara in December 1999. This variation reflects the variation often seen with Helicoverpa spp. in other traps which are not gender-specific, such as light traps. Whether it reflects a bias in the real sex ratio of the population, or just behavioural differences, is not clear.

We do not routinely run blank traps, but have done so on a few occasions and got no more than 0.01 moths/trap/day, even when moth numbers in the area are high, as estimated by pheromone traps. The moths in blank traps often tend to be males. We do not re-use pheromone traps for plant volatiles, but, given that we are rotating traps, we cannot exclude re-emission from pheromone residues around the trap site as the cause of a few male moths in the traps. However, we are confident that although moth numbers in the volatile traps are often low, the moths which are caught are generally responding to the volatile, not the trap itself.

These trials have been done at different times, and in different areas, different crops and different weather conditions. In some cases the conditions have been less than optimal. Examples include the Twynam 1, 2 and 3 trials in the Ord River, where night temperatures were often too low for moth activity, and the last two Wamara trials in 2000 where moth numbers in the district were extremely low. Because of these differences it is not possible to directly compare the numbers of moths caught between trials. We have therefore expressed the results as a percentage of the pheromone traps run in the same experiments. This percentage has been calculated on the catch interval with the highest volatile catches. We do this because the residual life of some volatiles is short and some formulations probably lose attractiveness during the course of the trials, whereas the pheromone release rate remains relatively constant. PBE1 was also included in almost all of these trials, as a standard plant volatile blend. The unweighted average catch of PBE1, across all trials, was 5.8% of the pheromone catch. Though PBE1 and some other volatile formulations occasionally caught up to 15-33% of the corresponding pheromone catch, when this occurred the overall numbers of moths were low, so the results are statistically unreliable.

No formulation was consistently better than PBE1. This included blends such as PF3Hs and 2 PF3Hs which performed better than PBE1 in the olfactometer (though these later-developed blends have only been tested once in the field). It also included the Texas (Lopez patent) blend; Though this performed better than our blends in one trial (Attleigh 2 chickpea), the pattern was not repeated in subsequent trials.

Phenylacetaldehyde on its own was tested in two early trials, and caught very few moths, mostly males. In a third direct comparison with PBE1, it caught less than a quarter of the PBE1 catch.

Another formulation which caught very little was the blend of Landolt (U.S. Pat. No. 6,190,653) which consists of acetic acid and some alcohols found in fermenting fruit. It works against Lacanobia subjuncta (a fruit-feeding noctuid) and bertha armyworm, Mamestra configurata.

TABLE 3
Summary of 21 field trapping trials

| | | | | | | Peak |
|---|---|---|---|---|---|---|
| | | | | | | catch |
| | | | | Whole | experiment % | % of |
| Trial | Location | Dates | Attractant | Lure N | female | phero |
| Wamara1Soybean | Downs | 1-30 Jan 99 | PAA | 10 | 10.0 | 0.4 |
| Wamara2 Soybean | Downs | 13-19 Feb 99 | PAA | 5 | 0.0 | 1.1 |
| Wamara3 Soybean | Downs | 20 Feb-12 Mar 99 | PAA | 8 | 62.5 | 3.1 |
| | | | PBE1 | 53 | 66.0 | 14.1 |
| Wamara4 Soybean | Downs | 13 Mar-8 Apr 99 | PBE1 | 15 | 86.6 | 1.9 |
| | | | PBEL | 8 | 37.5 | 5.8 |
| | | | PBEHs | 11 | 54.5 | 1.0 |
| Twynam1Cotton | Ord | 13-16 May 99 | PBE1 | 0 | NA | 0.0 |
| | | | PBE2 | 3 | 33.3 | 15.4 |
| Twynam2 Cotton | Ord | 18 May-1 Jun 99 | PBE1 | 0 | NA | 0.0 |
| | | | PBE2 | 2 | 50.0 | 2.4 |
| Twynam3 Cotton | Ord | 15-29 Jun 99 | PBE1 | 1 | 100.0 | 16.6 |
| | | | PBE2 | 1 | 100.0 | 16.6 |
| | | | PBE3 | 3 | 66.6 | 33.3 |
| | | | PBEHs | 4 | 75.0 | 16.6 |
| Henan Cotton | China | 17-26 Aug 99 | PBE1 | 12 | | 4.1 |
| | | | PN1 | 8 | | 2.7 |
| | | | PN4 | 12 | | 4.1 |
| | | | PopEx | 15 | | 5.2 |
| Tywnam4 Cotton | Ord | 10-24 Sep 99 | PBE1 | 6 | 33.3 | 33.3 |
| | | | PBEu | 3 | 0.0 | 5.0 |
| | | | PBBE | 6 | 66.6 | 10.0 |
| | | | PBBEu | 9 | 22.2 | 20.0 |
| Attleigh1 Wheat | Downs | 25 Sep-8 Oct | PBE1 | 2 | 50.0 | 8.7 |
| | | 99 | PB2P | 3 | 66.7 | 5.0 |
| Attleigh1 Chickpea | Downs | 25 Sep-8 Oct | PBE1 | 6 | 66.6 | 8.7 |
| | | 99 | PB2P | 4 | 75.0 | 1.7 |
| Attleigh2 Chickpea | Downs | 12-29 Oct 99 | PBE1 | 14 | 71.4 | 7.4 |
| | | | ChinB | 21 | 57.1 | 6.6 |
| | | | ChinC | 9 | 88.9 | 1.3 |
| | | | Texas | 33 | 90.9 | 10.7 |
| Wamara1 | Downs | 18-29 Nov 99 | PBE 1 | 5 | 80.0 | 2.8 |
| Dryland cotton | | | PB2PS | 6 | 16.7 | 1.6 |
| | | | ChinA | 16 | 43.8 | 4.4 |
| | | | Texas | 9 | 33.3 | 2.5 |
| Wamara2 | Downs | 30 Nov-7 Dec | PBE 1 | 6 | 16.7 | 5.1 |
| | | 99 | | | | |
| Dryland cotton | | | PB2PS | 11 | 36.4 | 2.2 |
| | | | PBEMb | 6 | 0.0 | 1.8 |
| | | | Texas | 12 | 25.0 | 5.3 |
| Wamara3 | Downs | 6-8 Dec 99 | PBE 1 | 25 | 8.0 | 1.3 |
| Sunflower | | | Texas | 37 | 5.4 | 3.1 |
| Wamara4 | Downs | 16-30 Dec 99 | PBE 1 | 11 | 18.2 | 2.2 |
| Dryland cotton | | | PBE 4 | 10 | 30.0 | 2.2 |
| | | | PBE 5 | 8 | 12.5 | 1.7 |
| | | | Texas | 7 | 42.9 | 2.1 |
| Wamara5 Soybean | Downs | 22 Feb-4 Mar | PBE 1 | 4 | 75.0 | 2.9 |
| | | 00 | Landolt's | 0 | NA | 0.0 |
| | | | PBE 1 + L's | 2 | 50.0 | 1.6 |
| Attleigh1 Chickpea | Downs | 28 Sep-11 Oct | PBE 1 | 21 | 33.3 | 1.8 |
| | | 00 | PF3Hs | 22 | 13.6 | 2.1 |
| | | | 2PF3Hs | 11 | 27.3 | 0.3 |
| | | | PF32P | 10 | 100.0 | 0.8 |
| Attleigh2 Chickpea | Downs | 12-23 Oct 00 | PBE/FA* | 10 | 60.0 | 5.0 |
| | | | PBE/FA/Z11:16OH* | 6 | 50.0 | 0.9 |
| | | | PBELo | 15 | 73.3 | 7.2 |
| | | | PBELo/Z11:16OH | 21 | 80.1 | 10.8 |
| Wamara1 | Downs | 24 Oct-6 Nov | PBE 1 | 5 | 20.0 | 4.0 |
| | | 00 | | | | |
| Ingard cotton | | | PBE 1/FA/Z11:16OH | 1 | 0.0 | 1.0 |
| | | | PBELo | 6 | 50.0 | 2.0 |
| | | | PBELo/FA/Z11:16OH | 2 | 0.0 | 1.6 |
| Wamara2 | Downs | 15-17 Nov 00 | PBE 1 | 9 | 56 | 7.4 |
| Ingard cotton | | | PBELo/16:OH | 6 | 33 | 2.5 |
| | | | PBELo | 11 | 55 | 2.0 |
| | | | PBELo/16:OHI/FA | 5 | 40 | 1.6 |

Attractant Blends:
PAA=Phenylacetaldehyde: 5% in Sirene
PBEL=2% phenylacetaldehyde, 2% benzyl alcohol, 2% methyl eugenol
PBE2=0.5% phenylacetaldehyde, 4% benzyl alcohol, 2% methyl eugenol
PBE 4=0.5% phenylacetaldehyde, 0.5% benzyl alcohol, 0.5% methyl eugenol
PBE 5=0.2% phenylacetaldehyde, 0.2% benzyl alcohol, 0.2% methyl eugenol
PBEMb=1% phenylacetaldehyde, 2% benzyl alcohol, 2% methyl eugenol, 2% 3-methyl-1-butanol
PBES=2% phenylacetaldehyde, 2% benzyl alcohol, 2% methyl eugenol, 2% Z-3-hexenyl salicylate
PB2PS=1% phenylacetaldehyde, 2% benzyl alcohol, 2% 2-phenylethanol, 2% Z-3-hexenyl salicylate
PN1=1% phenylacetaldehyde, 2% benzyl alcohol, 2% linalool, 1% Z-3-hexenol, 2% eugenol, 1% benzaldehyde, 5% 3-hydroxy-benzaldehyde
PN4=1% phenylacetaldehyde, 2% benzyl alcohol, 2% linalool, 1% Z-3-hexenol, 2% eugenol, 1% benzaldehyde, 5% 3-hydroxy-benzaldehyde, 2% (−)-trans-caryophyllene
PopEx Extract=steam-distilled extract of black poplar leaves (from Henan Agricultural University)
   PBEu=2% phenylacetaldehyde, 2% benzyl alcohol, 2% eugenol
   PBBE=2% phenylacetaldehyde, 2% benzyl alchol, 2% benzaldehyde, 2% methyl eugenol
   PBBEu=2% phenylacetaldehyde, 1% Benzyl alcohol, 1% benzaldehyde, 2% eugenol
   PB2P=1% phenylacetaldehyde, 2% benzyl alcohol, 2% 2-phenylethanol
   PF3Hs=1% phenylacetaldehyde, 1.4% a-pinene, 0.4% limonene, 1% cineole, 2% Z-3 hexenyl salicylate
   2 PF3Hs=2% 2-phenylethanol, 1.4% a-pinene, 0.4% limonene, 1% cineole, 2% Z-3-hexenyl salicylate
   PF32P=1% phenylacetaldehyde, 1.4% a-pinene, 0.4% limonene, 1% cineole, 2% 2-phenylethanol
   ChinA=50% phenylacetaldehyde, 20% eugenol, 17% 2-phenylethanol, 10% benzyl alcohol, 3% benazaldehyde
   ChinB=3.5% eugenol, 2.7% 2-phenylethanol, 1.3% benzyl alcohol, 0.5% benzaldehyde
   ChinC=4% 2-hydroxybenzaldehyde, 1.8% eugenol, 0.6% benzyl alcohol, 0.2% benzaldehyde, 1.4% 2-phenylethanol
   Landolt's=400 ml of 1% acetic acid+1 ml of neat 3-methyl-1-butanol in Eppendorf tube
   PBE 1+L's=PBE 1 on corflute+Landolt's in Eppendorf tube
   Texas=1.89% phenylacetaldehyde, 0.73% methyl salicylate, 1.96% limonene, 1.83% 2-phenylethanol, 1.59% methyl2-methoxybenzoate
   PBE/FA*=50 ul each of phenylacetaldehyde, benzyl alcohol and methyl eugenol mixed with 1.4 g evening primrose oil, 0.9 g palmitic acid, 0.29 g oleic acid and 2.5 g hexane
   PBE/FA/Z11:16OH*=PBE/FA+200 ul of Z11:16OH in hexane
   PBELo=2% phenylacetaldehyde, 2% benzyl alcohol, 2% methyl eugenol, 2% linalool
   PBELo/Z11:16OH=PBELo+200 ul of Z11:16OH
   PBE 1/FA/Z11:16OH=5 g of 2% PBE 1 Sirene+5 g of 5.6 g evening primrose oil, 3.7 g palmitic acid, 0.7 g oleic acid, 10 mg Z11:16OH
   PBELo/FA/Z11:16OH=5 g of 2% PBELo Sirene+5 g of 5.6 g evening primrose oil, 3.7 g palmitic acid, 0.7 g oleic acid, 10 mg Z11:16OH
   PBELo/Hexadecanol=5 g of 2% PBELo Sirene+100 mg hexadecanol
PBELo/Hexadecanol/FA=5 g of 2% PBELo Sirene+5 g fatty acid mixture+100 ul each of phenylacetaldehyde, benzyl alcohol, methyl eugenol linalool+200 mg hexadecanol
   Other species of moths caught in field trapping experiments

Some of our blends have also caught substantial numbers of other pest moth species. The catch of other moths appears to depend on the relative abundance of these species during the trial. During spring trials in the Darling Downs, most formulations have caught significant numbers of the native budworm, Helicoverpa punctigera, the false looper, Chrysodeixis argentifera, and the common armyworm Mythimna convecta. During summer and autumn on the Downs, large numbers of soybean loopers, Thysanoplusia orichalcea, were caught. Like the false looper, this species seems to respond to any blend containing phenylacetaldehyde. In trials in the Ord, the beet armyworm Spodoptera exigua was commonly caught. In a trial at Goondiwindi, large numbers of the cotton looper Anomis flava were caught.

Open Field Trials

Trial 1

This trial was conducted to determine whether the PF3Hs blend, in aqueous/oil formulation, would attract and kill Helicoverpa armigera moths when placed on plants in the open field. The blend of Lopez et al. (2000) was included for comparison, along with a blend containing no floral or leaf volatiles. All blends contained 0.5% methomyl. The trial was conducted on sweet corn near Bowen, Qld. The corn was in the vegetative stage, about 40 cm high, and planted in rows 75 cm apart which were oriented across the prevailing wind. The field was approximately 3 ha, and had not been treated with any insecticides prior to the experiment. Three rows, approximately 280 m long and 40 m apart, were selected for treatment. Each row was divided into 5 sections of 50 m each, with a 5 m buffer between sections. In each row, one section was treated with PF3Hs, another with the Lopez blend and a third with the no-volatile blend. The remaining two sections served as unsprayed controls. Two AgriSense pheromone traps were established in a similar adjacent field of corn to monitor H. armigera numbers.

The attractant blends (PF3Hs and Lopez) were formulated in canola oil (80 g) in water (372 ml) with the addition of sugar (320 g) Kemotan (sorbitan monostearate, emulsifying agent; 18 g), vitamin E (antioxidant; 0.8 g), BHT (2,6-di-tert-butyl-4-methylphenol, antioxidant; 0.8 g), Xanthan gum (thickener; 0.4 g) and blended. This produces a concentrate of about 750 ml. The attractant volatiles (total of 20 ml, in the proportions described previously, were then added and thoroughly mixed. To them, just before application, 760 ml of water, 40 ml of 20% technical grade methomyl dissolved in ethanol, and 8 ml of blue food colouring dye were added, to give a final volume of 1.5 litres. The mixture was then shaken. The final concentration of the attractant blends (total volatiles) was 1.33% w/v, and that of methomyl was 0.53% w/v. The no-volatiles blend was formulated in the same way, but omitting the volatiles.

The mixtures were applied by hand, shaken onto the plants from a bottle with a small aperture, at a rate of 500 ml per 50 m, in the late afternoon. Early on each of the next four mornings, the furrows between the rows were searched for dead moths. Five furrows upwind and five downwind of the treated row were searched, and all moths found were identified to species. Those of the most abundant species were dissected and sexed. The totals of moths found for the experiment, and the percentage of females, are shown in Table 4.

The most abundant noctuid in the area, as indicated by the counts of dead moths and by casual observations at nearby lights, was not H. armigera but the common armyworm, Mythimna convecta. The numbers of this species killed ranged from 79.3 to 107.7 per 50 m section in the sprayed sections, but there were no significant differences between the PF3Hs and Lopez treatments, and the formulation with no volatiles. There were also substantial numbers found in the unsprayed areas, but they were significantly lower than all the sprayed areas. M. convecta is known to respond to fermentation volatiles and to sugar alone (McDonald 1990). These results indicate that the PF3Hs and Lopez blends do not significantly add to that attraction. The presence of dead M. convecta moths in the unsprayed sections suggests that they either moved there after contacting the spray but before dying, or were blown there by wind, after death. No dead moths were found in a search of 500 m of furrows in the adjacent corn field, approximately 200 m away, suggesting that no factors apart from our treatments were causing mortality in the experimental field.

Numbers of H. armigera killed were lower. Pheromone catches in the adjacent field were low, ranging from 5.5 to 10 per trap per night during the experiment. This suggests that the numbers of this species active in the treated field were low. Nevertheless, between 4.3 and 14.3 moths were killed per 50 m for the sprayed treatments. The numbers in both the PF3Hs and Lopez treatments were significantly higher than in the treatment with no volatiles. The PF3Hs killed more than the Lopez blend, but the difference was not statistically significant. As with M. convecta, a gradient across the rows (the more downwind rows having the most dead moths) made analysis difficult, with high standard errors for many treatments. A few H. armigera were also found in unsprayed rows, to where they probably moved or were blown after contacting the sprays.

Other than H. armigera, the most common noctuids killed were Mythimna loreyimima (sugarcane armyworm), Spodoptera litura (cluster caterpillar) and Chrysodeixis spp. (false loopers). Significantly higher numbers of these were found in the sprayed compared to the unsprayed treatments. There were no significant differences between sprayed treatments, though the numbers were higher in the Lopez and PF3Hs than in the no-volatiles treatments.

For both M. convecta and H. armigera, the moths killed were predominantly females (between 69 and 81% in the sprayed treatments, for both species). In both cases the highest percentage of females was in the PF3Hs treatments, though the difference was only statistically significant for M. convecta, where larger numbers were available. This biased sex ratio suggests that the tendency for greater numbers of females compared to males, found previously in trapping studies, is not merely an artifact of including pheromones in those trials, but an intrinsic property of the attractants or the behaviour of moths in relation to them.

TABLE 4
Mean numbers of dead moths found per
50 m of treated row during the first open field trial.
M.c. = Mythimna convecta, H.a. = Helicoverpa armigera.
Within the same column, means bearing different letters
are significantly different (p < 0.05) using Fisher's
pairwise comparison test for the number of moths, and
Zar's multiple range test for the percent female.

| | |
|---|---|
| Numbers of | Percent |
| moths | female |

| Blend | M.c. | H.a. | Other | M.c. | H.a. |
|---|---|---|---|---|---|
| Unsprayed | 24.5a | 1.5a | 0.5a | 57a | 67a |
| No volatiles | 90.3b | 4.3a | 4.3b | 76c | 69a |
| Lopez | 107.7b | 10.0b | 6.0b | 69b | 70a |
| PF3Hs | 79.3b | 14.3b | 5.3b | 81c | 81a |

The numbers of moths killed over time is shown in FIG. 10. It is clear that the effects of all the formulations persisted over several nights. The steady decline in moths killed by the no-volatiles treatment probably represents the declining activity of methomyl, which is known to be a short-residual insecticide. In comparison, the numbers killed by the Lopez and PF3Hs blends declined more slowly over the first three days. This is particularly noticeable for the Lopez blend with M. convecta, and for the PF3Hs blend with H. armigera. It suggests that the volatiles persisted at levels high enough to attract moths over at least three nights. Though the droplets on the plants dried out during the first day after application, they re-absorbed moisture from dew on the second and subsequent nights. They would probably have been active in both contact and ingestion modes over all nights of the trial.

The spatial distribution of moths across the ten furrows (five upwind and five downwind) surrounding the treated rows is shown in FIG. 11. There were no obvious differences between sprayed treatments or moth species, so these data have been combined and compared with the unsprayed treatment for FIG. 11. While the adjacent furrows, especially the downwind one, had the greatest numbers, there were substantial numbers further away from the centre. This was especially so for the unsprayed treatment. It reinforces the conclusion, made from the finding of dead moths in unsprayed areas, that moths can move some distance after contacting the insecticide. It also suggests that the numbers of moths given in Table 4 are substantial underestimates of the number actually killed by the treatments.

This field trial had some design deficiencies, notably insufficient buffer zones due to the small field size, and inadequate control of the windward gradient in moth numbers. Nevertheless, it yielded results. Despite a low field population, substantial numbers of H. armigera were killed. It is likely that the numbers killed were greater than those recorded, because of movement of poisoned moths out of the treatment area, because the searching was not completely effective, and because predators (mostly ants) were known to remove some moths before collection.

Addition of PF3Hs and the Lopez blend to sugar/insecticide formulations significantly increased the number killed, and the PF3Hs blend was at least as good as the Lopez blend. There was evidence that the effects persisted over several nights, and the moths killed were mostly females.

Trial 2

This trial was intended to compare carbaryl and methomyl as toxicants for inclusion in attractant blends. It also repeated the comparison between the PF3Hs and Lopez blends, with another blend (PBELo) also included. The trial was conducted in sweet corn similar to that for the first trial, except that the field was larger (7 ha), which allowed for buffer zones to be increased to 17 m between sections. Another difference was that the field had been treated with methomyl 5 days before the experiment began. A few dead moths resulting from this spray were still present on the site, but they were easily distinguishable from moths killed during the experiment because they were dried out and often partially eaten by ants. They were not included in the results.

Formulations were made in the same way as the first trial. The PBELo formulation had the same total volatile concentration (1.33%) as the other formulations. Late on the first afternoon of the trial, these formulations were applied with 1% carbaryl added. On the following morning, the furrows were searched. Only five Helicoverpa armigera, one in each of the four volatile treatments and one in the unsprayed control, were recovered. No Mythimna convecta or other noctuids were found. The mean catch from two AgriSense pheromone traps in adjacent fields was 7.5 moths.

On the second day each section was re-treated with the same formulation, but using 0.5% methomyl as the toxicant. The cumulative mean numbers found in the different treatments 1, 2, 4 and 6 days after re-treatment are shown in Table 5, along with the cumulative mean pheromone catch from the two traps in adjacent corn fields. Numbers of M. convecta were much lower than in trial 1, probably reflecting the effects of the methomyl spray which preceded the experiment. This species was therefore included along with other noctuid moths in the “other” columns in Table 5. Numbers of H. armigera in pheromone traps were also quite low, averaging 6.25 per trap per night for a cumulative total of 37.5 per trap over the 6 days.

There was a general trend for more moths to be found in the sections treated with formulations containing volatiles. For H. armigera and for the category of other noctuids, PF3Hs killed significantly more moths on the first night than the no-volatiles treatment, or any of the other volatile treatments. Notably it killed significantly more than the Lopez blend. On subsequent days there were no statistically significant differences, and by the end of the trial the cumulative numbers killed by each of the volatile blends were very similar. This suggests the existence of one or more components of the PF3Hs blend which are especially attractive to H. armigera, but which dissipate quickly. The Lopez blend killed the highest numbers of other noctuid moths, though the only occasion on which this was statistically significant was compared to PBELo on day 2. The overall percentage females for H. armigera in each treatment are also shown in Table 5. They were lower than in trial 1, which may reflect behavioural differences between the sexes in relation to differences between the sites, especially the prior use of insecticide on the second site. There were no significant differences between treatments in the percentage of females.

TABLE 5
Cumulative mean numbers of dead moths
found per 50 m of treated row during the second open
field trial, and cumulative pheromone trap catches of
H. armigera. H.a. = Helicoverpa armigera, Other = other
noctuid species. Within the same column, means bearing
different letters are significantly different (p < 0.05)
using Fisher's pairwise comparison test.

| | 1 day | | 2 days | | 4 days | | 6 days | | |
|---|---|---|---|---|---|---|---|---|---|
| | Pheromone | | | | | | | | |
| | 4.5 | | 12.0 | | 25.0 | | 37.5 | | % Fem |
| Blend | Ha | Other | Ha | Other | Ha | Other | Ha | Other | Ha |
| Unsprayed | 0.7a | 0.7a | 1.3a | 2.0a | 1.7a | 5.6a | 6.3a | 6.3a | 36.3 |
| No volatiles | 2.7a | 2.0a | 8.0b | 8.7b | 12.7b | 15.8b | 23.7b | 23.7ab | 55.1 |
| Lopez | 3.7a | 7.0b | 11.7bc | 15.0c | 26.3c | 25.4c | 44.0c | 40.0b | 49.2 |
| PBELo | 3.3a | 2.7a | 10.0bc | 8.7b | 22.3c | 18.0bc | 42.3c | 32.0b | 50.3 |
| PF3Hs | 9.3b | 6.0b | 17.0c | 9.7bc | 32.3c | 17.4bc | 44.0c | 24.0b | 45.5 |

The failure of the first spray, using carbaryl, was probably due to the relatively lower insecticidal activity of that chemical compared to methomyl. It may not have killed the moths. Alternatively, and more likely in view of the laboratory feeding trials, it may have allowed moths to move too far away from the trial site, before death, to be recovered by our searching method. Nevertheless, the results of Trial 2, from the time when methomyl was used, support those of Trial 1. The numbers of H. armigera killed were substantial, in relation to the likely low population density as indicated by pheromone traps. The extended persistence of the volatile formulations, as indicated by the numbers of moths killed between days 4 and 6, is also encouraging.

### INDUSTRIAL APPLICABILITY

The present invention is useful in the control of insect pests.

### REFERENCES

The contents of the following documents are incorporated herein by reference:
Beerwinkle, K. R., Shaver, T. N., Lingren, P. D. and Raulston, J R. (1996) Free-choice olfactometer bioassay system for evaluating the attractiveness of plant volatiles to adult Helicoverpa zea. Southwestern Entomologist 21 395-405.
Cunningham, J. P., West, S. A. and Wright, D. J. (1998) Learning in the nectar foraging behaviour of Helicoverpa armigera. Bulletin of Entomological Research 23, 363-369.
Ditman, L. P. (1937) Observations on poison baits for corn earworm control. Journal of Economic Entomology 30, 116-118.
Gregg, P. C. (1993) Pollen as a marker for Helicoverpa armigera (Hubner) and H. punctigera Wallengren (Lepidoptera: Noctuidae) emigrating from western Queensland. Australian Journal of Ecology 18, 209-219.
Gregg, P. C. and Wilson, A. G. L. (1991) Trapping methods for adults. In Zalucki, M. P. (ed.) Heliothis: Research methods and prospects. Springer-Verlag, Berlin, pp.30-48.
Gregg, P. C., Del Socorro, A. P., Henderson, G. S., Forrester, N. W. and Moore, C. (1998) Plant-based attractants for adult Helicoverpa spp. In Zalucki, M. P., Drew, R. A. I. and White, G. G. (eds.) Pest management—future challenges. Proceedings of the Sixth Australasian Applied Entomological Research Conference. University of Queensland, Brisbane. pp. 342-348.
Landolt, P. J., Lenczewski, B. and Heath, R. R. (1991) Lure and toxicant system for the cabbage looper (Lepidoptera: Noctuidae). Journal of Economic Entomology 84, 1344-1347.
Lopez, J. D., Shaver, T. N., Beerwinkle, K. R. and Lingren, P. D. (2000) Feeding attractant and stimulant for adult control of noctuid and/or other Lepidopteran species. U.S. Pat. No. 6,074,634.
Mafra-Neto, A. and Habib, M. (1996) Evidence that mass trapping suppresses pink bollworm populations in cotton fields. Entomologia Experimentalis et Applicata 81, 315-323.
McDonald, G. (1990) A fermentation trap for selectively monitoring activity of Mythimna convecta (Walker) (Lepidoptera:Noctuidae). Journal of the Australian Entomological Society 29, 107-108.
Plepys, D. (2000) Behavioural and electrophysiological responses of the silver Y moth Autographa gamma (Lepidoptera: Noctuidae) to floral volatiles. Abstracts of the XXI International Congress of Entomology, Foz do Iguassu, Brazil, 20-26 August 2000. Vol. 1, p. 180.
Smith, J. W., McJibbern, G. H., Villavaso, E. J., McGovern, W. L. and Jones, R. G. (1994) Management of the cotton boll weevil with attract-and-kill devices. In Constable, G. A. and Forrester, N. W. (eds.) Challenging the future. Proceedings of the World Cotton Research Conference I. CSIRO, Canberra, pp. 480-484.

1. A composition for attracting noctuid moths comprising one or more floral volatiles selected from the group consisting of phenylacetaldehyde, 2-phenylethanol, benzyl alcohol and a lilac aldehyde in admixture with one or more leaf volatiles selected from the group consisting of geraniol, 3-carene, limonene, Z-3-hexenyl acetate, Z-3-hexenyl salicylate, linalool, α-pinene and cineole, and an inert carrier.

2. A composition as claimed in claim 1 wherein a single floral volatile is present and it is phenylacetaldehyde.

3. A composition as claimed in claim 2 comprising phenylacetaldehyde in admixture with limonene, linalool and cineole.

4. A composition as claimed in claim 3 further Comprising Z-3-hexenyl salicylate or gamma-terpinene.

5. A composition as claimed in claim 1 wherein an admixture of floral volatiles comprising phenylacetaldehyde and 2-phenylethanol and/or benzyl alcohol is present.

6. A composition as claimed in claim 5 further comprising an admixture of limonene, linalool and cineole.

7. A composition as claimed in claim 5 further comprising z-3-hexenyl salicylate.

8. A composition as claimed in any one of claims 1 to 7 wherein the inert carrier is selected from the group consisting of polyols, esters, methylene chloride, alcohol, preferably C₁-C₄alcohol, vegetable oil or SIRENE.

9. A composition as claimed in any one of claims 1 to 8 further comprising an insect toxicant.

10. A composition as claimed in claim 9 wherein the insect toxicant is a pyrethroid or a carbamate.

11. A composition as claimed in claim 10 wherein the insect toxicant is selected from the group consisting of bifenthrin, carbaryl, methomyl, acephate, thiodicarb, cyfluthrin, malathion, chlorpyrifos, emamectin benzoate, abamectin, spinosad, endosulfan, and mixtures thereof.

12. A composition as claimed in any one of claims 1 to 8 further comprising a bacterial or viral pathogen.

13. A composition as claimed in any one of claims 1 to 8 further comprising an insect growth regulator or a compound capable of eliciting behaviour modification or disrupting physiological functions.

14. A composition as claimed in any one of claims 1 to 13 further comprising a feeding stimulant and/or food source.

15. A composition as claimed in any one of claims 1 to 14 further comprising humectants, preservatives, thickeners, antimicrobial agents, antioxidants, emulsifiers, film-forming polymers and mixtures thereof.

16. A method of attracting noctuid moths to a locus, comprising the steps of applying a composition as claimed in any one of claims 1 to 15 to said locus.

17. A method as claimed in claim 16 wherein the locus is a trap crop.

18. A method as claimed in claim 17 wherein the composition is applied to the trap crop, preferably by spraying.

19. A method as claimed in claim 18 wherein the composition in disseminated from a dispenser located within the trap crop.

20. A method as claimed in claim 16 wherein said locus is an insect trap.

21. The use of Z-3-hexenyl salicylate as an attractant for noctuid moths.
